(19) United States
(12) Patent Application Publication
(10) Pub. No.: US 2025/0041305 A1
(43) Pub. Date: Feb. 6, 2025

(54) PHARMACEUTICAL COMPOSITION OF SEPIAPTERIN

(71) Applicant: PTC Therapeutics, Inc., South Plainfield, NJ (US)

(72) Inventors: Akm Nasir UDDIN, Somerset, NJ (US); Mandar Vasant DALI, Bridgewater, NJ (US); Dhaval PATEL, Bridgewater, NJ (US)

(21) Appl. No.: 18/696,567

(22) PCT Filed: Sep. 29, 2022
(86) PCT No.: PCT/US2022/045214

§ 371 Date: Mar. 28, 2024

Related U.S. Application Data
(60) Provisional application No. 63/250,167, filed on Sep. 29, 2021.

(51) Int. Cl. A61K 31/519; A61K 9/00; A61K 9/16

(52) U.S. Cl. CPC A61K 31/519; A61K 9/009; A61K 9/1617; A61K 9/1623; A61K 9/1635; A61K 9/1652; A61K 9/1682

(57) ABSTRACT

This invention discloses a pharmaceutical composition of sepiapterin with improved processability, stability, and palatability.

### CROSS-REFERENCE TO RELATED APPLICATION

This application claims priority to U.S. Provisional Application No. 63/250,167, filed Sep. 29, 2021, the contents of which are incorporated by reference herein.

### FIELD OF THE INVENTION

The invention generally relates to a pharmaceutical composition of sepiapterin, e.g., that has improved processability and stability relative to alternative pharmaceutical compositions of sepiapterin, and methods of making and using the same.

### BACKGROUND

Multiple disorders are associated with deficient or low tetrahydrobiopterin (BH₄) concentrations (e.g., Primary Tetrahydrobiopterin Deficiency, Phenylketonuria, and diabetic gastroparesis). These disorders may result in impaired hydroxylation of phenylalanine (Phe) to tyrosine (Tyr) resulting in hyperphenylalaninemia (HPA) or impaired hydroxylation of Tyr or tryptophan (Trp) or impaired synthesis of nitric oxide (NO) resulting in deficient neurotransmitter production (e.g., dopamine, serotonin, and NO).

Sepiapterin is a naturally occurring small molecule that serves as a substrate for de novo synthesis of BH₄ via the pterin salvage pathway making sepiapterin a naturally occurring precursor for BH₄. BH₄ is an essential cofactor for enzymes including PAH, tyrosine hydroxylase, tryptophan hydroxylase, fatty acid glyceryl ether oxygenase, and NO synthase.

Treatment with synthetic BH₄ (sapropterin dihydrochloride) is limited by poor bioavailability. Uptake of orally administered synthetic BH₄ has been shown to be hampered by rapid oxidation to 7,8-dihydrobiopterin (BH₂) and poor penetration into cells, resulting in a very low level of incorporation. In contrast, sepiapterin provides an exogenous source of sepiapterin that is stable in plasma, actively transported into cells, and converted to intracellular BH₄ by a simple, rapid unidirectional 2 step enzymatic reduction process as part of the pterin salvage pathway.

Sepiapterin is known to degrade in the presence of moisture, oxygen, and sunlight. Furthermore, known compositions of sepiapterin suffer from poor process efficiency, flow characteristics, and stability. Thus, a need exists for new pharmaceutical compositions of sepiapterin.

### SUMMARY OF THE INVENTION

In one aspect, the invention features a solid pharmaceutical composition comprising sepiapterin, a water-soluble diluent, a water-insoluble diluent, a disintegrant, a suspending agent, a glidant, a sweetener, and a lubricant.

In some embodiments, the sepiapterin is crystalline, e.g., crystalline Form F or Form D. In other embodiments, the crystalline sepiapterin is crystalline Form A, B, C, E, or G.

In some embodiments, the pharmaceutical composition is a powder.

In some embodiments, the pharmaceutical composition includes less than 60% humidity.

In some embodiments, the pharmaceutical composition is packaged in a sachet.

In some embodiments, the pharmaceutical composition includes about 20 to 50% sepiapterin by weight, e.g., about 20 to 30% sepiapterin by weight, about 22 to 28% sepiapterin by weight, about 24 to 26% by weight. In some embodiments, the pharmaceutical composition includes about 25% sepiapterin by weight.

In some embodiments, the diluents are selected from lactose, glucose, mannitol, xylitol, maltitol, sorbitol, isomalt, cellulose (e.g., crystalline or microcrystalline), sucrose, fructose, maltose, and trehalose. In some embodiments, the pharmaceutical composition includes two or more diluents, e.g., selected from lactose, glucose, mannitol, xylitol, maltitol, sorbitol, isomalt, cellulose (e.g., crystalline or microcrystalline), sucrose, fructose, maltose, and trehalose. In some embodiments, the diluents are soluble in water. In some embodiments, the diluents are insoluble in water.

In some embodiments, the pharmaceutical composition includes about 5 to 65% water-soluble diluent by weight, e.g., about 40 to 60% water soluble diluent by weight, about 45% to 55% water-soluble diluent by weight. In some embodiments, the pharmaceutical composition includes about 50% water-soluble diluent by weight.

In some embodiments, one water-soluble diluent is isomalt. In some embodiments, the pharmaceutical composition includes about 5 to 45% isomalt by weight, e.g., about 35 to 45% isomalt by weight. In some embodiments, the composition includes about 40% isomalt by weight.

In some embodiments, one water-soluble diluent is mannitol. In some embodiments, the pharmaceutical composition includes about 5 to 20% mannitol by weight, e.g., about 5 to 15% mannitol by weight. In some embodiments, the pharmaceutical composition includes about 10% mannitol by weight.

In some embodiments, one water-insoluble diluent is microcrystalline cellulose. In some embodiments, the composition includes about 15 to 39% microcrystalline cellulose by weight, e.g., about 17 to 25% microcrystalline cellulose by weight, about 19 to 23% microcrystalline cellulose by weight. In some embodiments, the composition includes about 21% microcrystalline cellulose by weight.

In some embodiments, the composition includes isomalt, mannitol, and microcrystalline cellulose.

In some embodiments, the disintegrant is selected from sodium starch glycolate, crospovidone, cross-linked alginic acid, crosslinked starch, crosslinked alginate sodium, carmellose, carmellose calcium, croscarmellose sodium, glycerin fatty acid ester, low-substituted sodium carboxymethyl starch, and partially pregelatinized starch. In some embodiments, the composition includes about 0.5 to 1.5% disintegrant by weight. In some embodiments, the disintegrant is croscarmellose sodium. In some embodiments, the pharmaceutical composition includes about 0.5 to 1.5% croscarmellose sodium by weight. In some embodiments, the pharmaceutical composition includes about 1% croscarmellose sodium by weight.

In some embodiments, the glidant is selected from silicon dioxide (e.g., colloidal), hydrated sodium sulfoaluminate, and talc. In some embodiments, the pharmaceutical composition includes about 0.2% to 0.6% glidant by weight. In some embodiments, the glidant is colloidal silicon dioxide. In some embodiments, the pharmaceutical composition includes about 0.2% to 0.6% colloidal silicon dioxide by weight. In some embodiments, the pharmaceutical composition includes about 0.4% colloidal silicon dioxide by weight.

In some embodiments, the sweetener is selected from sucralose, sodium saccharin, aspartame, and neutrame (with aspartame contraindicated in the treatment of hyperphenylalaninemia). In some embodiments, the pharmaceutical composition includes about 0.5% to 2.0% sweetener by weight. In some embodiments, the sweetener is sucralose. In some embodiments, the pharmaceutical composition includes about 0.5% to 2.0% sucralose by weight. In some embodiments, the pharmaceutical composition includes about 1.0% sucralose by weight.

In some embodiments, the lubricant is selected from glyceryl behenate, glyceryl behaptate, sodium stearyl fumarate, stearic acid, magnesium stearate, calcium stearate, sodium stearate, hydrogenated vegetable oils, colloidal silica, talc, waxes, boric acid, sodium benzoate, sodium acetate, sodium fumarate, sodium chloride, DL-leucine, polyethylene glycol, sodium oleate, sodium lauryl sulfate, and magnesium lauryl sulfate. In some embodiments, the pharmaceutical composition includes about 0.4% to 0.8% lubricant by weight. In some embodiments, the lubricant is magnesium stearate. In some embodiments, the pharmaceutical composition includes about 0.4% to 0.8% magnesium stearate by weight. In some embodiments, the composition includes about 0.6% magnesium stearate by weight.

In some embodiments, the suspending agent is selected from xanthan gum and hydroxyethyl cellulose. In some embodiments, the pharmaceutical composition includes about 0.5% to 2.0% suspending agent by weight. In some embodiments, the suspending agent is xanthan gum. In some embodiments, the pharmaceutical composition includes about 0.5% to 2% xanthan gum by weight. In some embodiments, the pharmaceutical composition includes about 1% xanthan gum by weight.

In one embodiment, the pharmaceutical composition includes about 25% sepiapterin, about 21% microcrystalline cellulose, about 40% isomalt, about 10% mannitol, about 1% croscarmellose sodium, about 1% xanthan gum, about 0.4% colloidal silicon dioxide, about 1.0% sucralose, and about 0.6% magnesium stearate by weight.

In another embodiment, the present invention relates to a method of preparing any of the foregoing pharmaceutical compositions, comprising steps of (1) sieving sepiapterin and at least one water-soluble diluent, water-insoluble diluent, disintegrant, suspending agent, glidant, and sweetener, and blending, forming a pre-blended mixture, (2) sieving a lubricant, (3) mixing the lubricant with the preblended mixture to form a lubricated preblended mixture, (4) preparing ribbons of the lubricated preblended mixture using a roller compactor, (5) milling the ribbons to form a milled formulation, (6) blending the milled formulation with a blender, thereby forming the pharmaceutical composition. In some embodiments, the method further includes filling the pharmaceutical composition into sachets.

In another embodiment, the present invention relates to a method of preparing a liquid formulation including dispersing any of the foregoing pharmaceutical compositions into a liquid. In some embodiments, the liquid is water or fruit juice. In some embodiments, the liquid formulation is a suspension.

### BRIEF DESCRIPTION OF THE DRAWINGS

FIG. 1 is a graph showing the dynamic vapor sorption curves for the sepiapterin drug substance.

FIG. 2 is a graph showing the dynamic vapor sorption curves for a formulation of the invention.

FIG. 3 is a graph showing the dissolution profiles of formulations of sepiapterin powder for oral use.

FIG. 4 is a schematic showing the design of a Phase 1, randomized, open-label, crossover study to evaluate the relative oral bioavailability of two formulations of sepiapterin.

FIG. 5A is a graph of mean concentration of BH₄ in ng/ml in human subjects after administration of 20 mg/kg of sepiapterin from Formula A (open squares) or from Formula B (solid triangles) over time.

FIG. 5B is a graph of mean concentration of BH₄ in ng/ml in human subjects after administration of 60 mg/kg of sepiapterin from Formula A (open circles) or from Formula B (solid squares) over time.

FIG. 6A is a graph of mean concentration of BH₄ in ng/ml in human subjects after administration of 20 mg/kg of sepiapterin from Formula A (open squares) or from Formula B (solid triangles) over time, where the data has been baseline adjusted.

FIG. 6B is a graph of mean concentration of BH₄ in ng/ml in human subjects after administration of 60 mg/kg of sepiapterin from Formula A (open circles) or from Formula B (solid squares) over time, where the data has been baseline adjusted.

### DETAILED DESCRIPTION OF THE INVENTION

The present inventors have discovered pharmaceutical compositions of sepiapterin comprising a water-soluble diluent, a water-insoluble diluent, a disintegrant, a suspending agent, a glidant, a sweetener, and a lubricant. The compositions disclosed herein provide improved processability and stability relative to alternative compositions of sepiapterin.

Compositions of sepiapterin of the present invention preferably have at least one of the following characteristics:
Rapid dispersion in aqueous solution (less than 1 minute).
Good processability properties.
Stability of sepiapterin in the composition.
Good bioavailability of sepiapterin when administered to a subject.

Pharmaceutical compositions of the invention have superior processability properties relative to alternative pharmaceutical compositions. Nonlimiting examples of processability qualities possessed by the pharmaceutical composition of the invention include good screw speed, no roll stickiness in a roller compactor, the formation of a ribbon during roller compaction that is continuous rather than broken or broken and brittle, a very hard ribbon, fair/passable flow, and uniform dispersion in water in less than 30 seconds. Pharmaceutical compositions of the invention have superior stability relative to alternative pharmaceutical compositions. In some embodiments, the pharmaceutical compositions of the invention are stable for at least 6 months at 2-8° C. and 25° C./60% relative humidity. Sepiapterin is prone to oxidative degradation, so the stability of sepiapterin is important.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, the materials, methods, and examples are illustrative only and not intended to be limiting. Other features of the disclosure are apparent from the following detailed description and the claims.

Titles or subtitles may be used in the specification for the sole convenience of the reader but are not intended to influence the scope of the present disclosure or to limit any aspect of the disclosure to any subsection, subtitle, or paragraph.

### Definitions

As used herein, the singular forms “a,” “an,” and “the,” are intended to include the plural forms as well, unless the context clearly indicates otherwise.

As used herein and in the claims, the phrase “at least one,” in reference to a list of one or more elements, should be understood to mean at least one element selected from any one or more of the elements in the list of elements, but not necessarily including at least one of each and every element specifically listed within the list of elements, and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 10%. In certain aspects, the term “about” is used to modify a numerical value above and below the stated value by a variance of 10%.

As used herein, the term “BH₄ related disorder,” refers to any disease or disorder that may derive a therapeutic benefit from modulation (e.g., inhibition) of the level of BH₄, e.g., phenylketonuria.

As used herein, the terms “subject” or “patient” are used interchangeably to refer to an individual human suffering from a disease described herein (e.g., phenylketonuria) that can be treated by administration of a composition described herein.

When a range of values is listed herein, it is intended to encompass each value and sub-range within that range. For example, “1-5 ng” or a range of “1 ng to 5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein, the terms “treat,” “treatment,” “treating” refer to therapeutic treatments, wherein the object is to reverse, alleviate, ameliorate, inhibit, slow down or stop the progression or severity of a disorder. The term “treating” includes reducing or alleviating at least one adverse effect or symptom of a condition, disease, or disorder. Treatment is generally “effective” if one or more symptoms or clinical markers are reduced. Alternatively, treatment is “effective” if the progression of a disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “excipient” as used herein means any substance, not itself a therapeutic agent, used as a carrier or vehicle for delivery of a therapeutic agent to a subject or added to a pharmaceutical composition to improve its handling or storage properties or to permit or facilitate formation of the dose unit of the composition into a discrete article, such as a capsule or tablet suitable for oral administration. Excipients include, by way of illustration and not limitation, diluents, disintegrants, binding agents, adhesives, surfactants, lubricants, glidants, surface modifying agents, substances added to mask or counteract a disagreeable taste or odor, flavors, dyes, fragrances, and substances added to improve the appearance of the composition.

As used herein, “administer” or “administration” refers to the act of physically delivering a substance as it exists outside the body into a subject.

### Sepiapterin

The pharmaceutical compositions of the invention include sepiapterin. In some embodiments of any of the foregoing compositions, sepiapterin is in crystalline form, e.g., Form A, B, C, D, E, F, or G as described in WO 2018/102314 and WO 2018/102315, the crystalline forms of which are hereby incorporated by reference.

In some embodiments, the crystalline form of sepiapterin is Form F of sepiapterin free base. Form F is characterized by refractions at angles of refraction 20 of at least about 9.7°, about 10.2°, about 11.3°, about 14.0°, about 14.6°, about 19.9°, about 22.2°, about 25.3°, and about 32.4°. In an essentially pure material of Form F of sepiapterin free base, peaks can be observed at angles of refraction 20 as set forth in Table 2.

TABLE 2

| Position [2θ°] | Relative Intensity |
|---|---|
| 9.7 | 98.27 |
| 10.2 | 100.00 |
| 11.3 | 22.47 |
| 14.0 | 5.01 |
| 14.6 | 12.36 |
| 19.9 | 5.63 |
| 21.1 | 3.72 |
| 22.2 | 5.37 |
| 22.7 | 4.04 |
| 24.5 | 2.99 |
| 25.3 | 17.65 |
| 27.2 | 3.10 |
| 32.4 | 5.29 |
| 36.7 | 2.72 |

In some embodiments, the crystalline form is Form D of sepiapterin free base. Form D is characterized by refractions at angles of refraction 20 of at least about 8.9°, about 10.3°, about 10.9°, about 17.8°, about 24.9°, about 26.0°, about 26.7°, about 26.8°, and about 28.3°. In an essentially pure material of Form D of sepiapterin free base, peaks can be observed at angles of refraction 20 as set forth in Table 3.

TABLE 3

| Position [2θ°] | Relative Intensity |
|---|---|
| 8.9 | 100.00 |
| 10.3 | 49.92 |
| 10.9 | 19.96 |
| 11.6 | 2.15 |
| 13.6 | 2.99 |
| 14.2 | 3.45 |
| 14.8 | 2.35 |
| 15.4 | 2.59 |
| 16.4 | 1.55 |
| 17.2 | 2.33 |
| 17.8 | 6.24 |
| 19.6 | 2.62 |
| 20.1 | 2.28 |
| 20.5 | 3.09 |
| 20.8 | 2.27 |
| 21.3 | 3.60 |
| 22.3 | 4.79 |
| 23.7 | 4.31 |
| 24.9 | 5.19 |
| 26.0 | 41.94 |
| 26.7 | 8.58 |
| 26.8 | 9.17 |
| 27.4 | 3.98 |
| 28.3 | 4.75 |
| 28.7 | 6.60 |
| 29.8 | 3.03 |
| 31.8 | 2.72 |
| 33.0 | 2.03 |
| 35.5 | 1.57 |
| 37.1 | 1.09 |

In some embodiments, the crystalline form is Form A of sepiapterin free base. Form A is characterized by the 20 peak positions of at least about 4.7°, about 7.4°, about 9.5°, about 11.3°, about 15.6°, about 26.2°, and about 27.2°. In an essentially pure material of Form A of sepiapterin free base, peaks can be observed at angles of refraction 20 as set forth in Table 4.

TABLE 4

| Position [2θ°] | Relative Intensity |
|---|---|
| 4.7 | 47.76 |
| 7.4 | 100.00 |
| 9.5 | 33.54 |
| 11.3 | 19.31 |
| 12.4 | 8.49 |
| 13.4 | 3.60 |
| 14.2 | 8.24 |
| 15.6 | 15.08 |
| 16.4 | 11.97 |
| 17.6 | 8.35 |
| 18.4 | 5.03 |
| 19.8 | 9.18 |
| 21.5 | 5.44 |
| 24.4 | 5.56 |
| 26.2 | 35.37 |
| 27.2 | 19.11 |
| 28.9 | 5.93 |

In some embodiments, the crystalline form is Form B of sepiapterin free base. Form B is characterized by refractions at angles of refraction 20 of at least about 8.4°, about 14.9°, about 16.9°, about 25.4°, and about 34.1°. In an essentially pure material of Form B of sepiapterin free base, peaks can be observed at angles of refraction 20 as set forth in Table 5.

TABLE 5

| Position [2θ°] | Relative Intensity |
|---|---|
| 8.4 | 100.00 |
| 14.9 | 2.34 |
| 16.9 | 10.70 |
| 25.4 | 84.90 |
| 34.1 | 3.00 |

In some embodiments, the crystalline form is Form C of sepiapterin free base. Form C is characterized by refractions at angles of refraction 20 of at least about 5.7°, about 7.8°, about 9.1°, about 11.5°, about 15.3°, about 16.0°, about 20.1°, about 25.4°, and about 26.6°. In an essentially pure material of Form C of sepiapterin free base, peaks can be observed at angles of refraction 20 as set forth in Table 6.

TABLE 6

| Position [2θ°] | Relative Intensity |
|---|---|
| 5.7 | 48.91 |
| 7.8 | 100.00 |
| 9.1 | 59.49 |
| 10.4 | 8.72 |
| 11.5 | 24.53 |
| 12.9 | 8.50 |
| 14.8 | 9.24 |
| 15.3 | 12.53 |
| 16.0 | 14.09 |
| 17.2 | 7.22 |
| 18.2 | 4.25 |
| 19.2 | 5.78 |
| 20.1 | 14.54 |
| 21.5 | 6.47 |
| 22.9 | 6.85 |
| 23.7 | 4.80 |
| 25.4 | 65.68 |
| 26.6 | 14.53 |
| 27.4 | 8.39 |
| 31.5 | 3.74 |
| 34.2 | 4.36 |

In some embodiments, the crystalline form is Form E of sepiapterin free base. Form E is characterized by refractions at angles of refraction 20 of at least about 6.0°, about 10.6°, about 12.1°, about 15.9°, about 20.9°, and about 24.6°. In an essentially pure material of Form E of sepiapterin free base, peaks can be observed at angles of refraction 20 as set forth in Table 7.

TABLE 7

| Position [2θ°] | Relative Intensity |
|---|---|
| 6.0 | 100.00 |
| 10.6 | 20.78 |
| 12.1 | 31.95 |
| 15.9 | 12.83 |
| 18.1 | 3.39 |
| 20.9 | 11.63 |
| 22.1 | 2.79 |
| 24.6 | 8.28 |
| 26.1 | 0.88 |
| 28.1 | 7.33 |
| 28.9 | 3.77 |
| 32.1 | 3.57 |
| 37.0 | 1.03 |

In some embodiments, the crystalline form is crystalline Form G of sepiapterin free base. Form G is characterized by refractions at angles of refraction 20 of at least about 10.0°, about 10.6°, about 11.2°, about 15.3°, about 15.9°, about 22.8°, about 24.4°, about 25.0°, about 25.7°, and about 26.6°. In an essentially pure material of the Form G of sepiapterin free base, peaks can be observed at angles of refraction 20 as set forth in Table 8.

TABLE 8

| Position [2θ°] | Relative Intensity |
|---|---|
| 5.3 | 8.30 |
| 6.9 | 4.54 |
| 10.0 | 100.00 |
| 10.6 | 69.64 |
| 11.2 | 6.59 |
| 13.5 | 7.52 |
| 15.3 | 26.59 |
| 15.9 | 26.43 |
| 16.0 | 23.41 |
| 16.9 | 4.28 |
| 18.6 | 13.02 |
| 19.3 | 11.90 |
| 20.1 | 7.22 |
| 20.8 | 11.01 |
| 22.8 | 16.77 |
| 23.5 | 19.60 |
| 24.4 | 41.45 |
| 25.0 | 23.99 |
| 25.7 | 65.40 |
| 26.6 | 39.64 |
| 27.6 | 13.04 |
| 28.7 | 6.55 |
| 30.8 | 14.76 |
| 32.2 | 9.63 |
| 33.7 | 5.16 |
| 37.5 | 5.80 |

In some embodiments, a pharmaceutical composition of the invention includes about 20-50% sepiapterin by total weight, e.g., about 20 to 40% sepiapterin by weight, about 20 to 35% sepiapterin by weight, about 20 to 30% sepiapterin by weight, about 22 to 28% sepiapterin by weight, about 24 to 26% by weight. In some embodiments, a pharmaceutical composition of the invention may include about 20% sepiapterin by weight, about 25% sepiapterin by weight, about 30% sepiapterin by weight, about 35% sepiapterin by weight, about 40% sepiapterin by weight, about 45% sepiapterin by weight, about 50% sepiapterin by weight.

### Excipients

In some embodiments, the pharmaceutical compositions of sepiapterin disclosed herein may include excipients such as at least one diluent, a disintegrant, a glidant, a suspending agent, a sweetener, an antioxidant, and a lubricant. Suitable excipients for use in the pharmaceutical compositions of the present invention are found in Remington: The Science and Practice of Pharmacy, (22nd ed.) ed. L. V. Allen, Jr., 2013, Pharmaceutical Press, Philadelphia, PA.

### Diluent

Pharmaceutical compositions may include a diluent, which may impart stability or improved moldability. Examples of diluents include cellulose (e.g., crystalline or microcrystalline), sugars such as lactose or glucose, sugar alcohols such as mannitol, xylitol, maltitol, sorbitol, and isomalt. More than one diluent may be employed.

Diluents may be soluble in water, e.g., glucose, lactose, isomalt, and mannitol, or insoluble in water, e.g., cellulose (crystalline or microcrystalline). Both water-soluble and water-insoluble diluents may be employed in the same composition.

The pharmaceutical composition may include about 10 to 85% diluent by weight, e.g., about 50 to 85% diluent by weight, about 60 to 80% diluent by weight, about 65 to 75% diluent by weight. In some embodiments, the pharmaceutical composition includes about 71% diluent by weight.

In some embodiments, the pharmaceutical composition includes a water-insoluble diluent selected from crystalline cellulose and microcrystalline cellulose, and at least one water-soluble diluent selected from lactose, glucose, mannitol, xylitol, maltitol, sorbitol, isomalt, sucrose, fructose, maltose, and trehalose. In some embodiments, the pharmaceutical composition includes microcrystalline cellulose and at least two water soluble diluents. In some embodiments the water-insoluble diluent and water-soluble diluent or combination of water-soluble diluents are present in a ratio of about 1:1 to about 1:4, about 1:2 to about 1:3, or about 1:2.5.

In one embodiment of the pharmaceutical compositions of the invention, the diluents are mannitol, isomalt, and microcrystalline cellulose.

The pharmaceutical composition may include about 5 to 65% water-soluble diluent by weight, e.g., about 40 to 60% water soluble diluent by weight, about 45% to 55% water-soluble diluent by weight. In some embodiments, the pharmaceutical composition includes about 50% water-soluble diluent by weight.

The pharmaceutical composition may include about 5 to 45% isomalt by weight, e.g., about 35 to 45% isomalt by weight. In some embodiments, the composition includes about 40% isomalt by weight.

The pharmaceutical composition may include about 5 to 20% mannitol by weight, e.g., about 5 to 15% mannitol by weight, about 5 to 10% mannitol by weight. In some embodiments, the pharmaceutical composition includes about 10% mannitol by weight.

In some embodiments, pharmaceutical compositions that include high levels of mannitol (e.g., >10% mannitol) adhered to rollers during roller compaction, resulting in broken ribbons.

The pharmaceutical composition may include about 15 to 40% water-insoluble diluent by weight, e.g., about 15 to 30% water-insoluble diluent by weight, about 15 to 25% water-insoluble diluent by weight. In some embodiments, the composition includes about 21% water insoluble diluent by weight.

The composition may include about 15 to 40% microcrystalline cellulose by weight, e.g., about 17 to 25% microcrystalline cellulose by weight, about 19 to 23% microcrystalline cellulose by weight. In some embodiments, the composition includes about 21% microcrystalline cellulose by weight.

In some embodiments, isomalt, mannitol, and microcrystalline cellulose provide compositions of the invention with superior sedimentation properties, dose recovery, and mouthfeel after reconstitution in a liquid (e.g., water or fruit juice) than pharmaceutical compositions formulated with alternative diluents or proportions of isomalt, mannitol, and microcrystalline cellulose other than the proportions described above.

### Suspending Agent

A pharmaceutical composition may include a suspending agent. In some embodiments, the pharmaceutical compositions disclosed herein are dispersed in a liquid prior to administration to a subject. The inclusion of a suspending agent prevents sedimentation of the pharmaceutical composition when dispersed in a liquid. Exemplary suspending agents include agar, alginic acid, sodium carboxymethyl cellulose, carrageenan, dextrin, gelatin, guar gum, hydroxyethyl cellulose, hydroxypropyl cellulose, hypromellose, methyl cellulose, polyethylene glycol, povidone, tragacanth, or xanthan gum.

The composition may include about 0.5% to 2.0% suspending agent by weight.

In one embodiment of the pharmaceutical compositions of the invention, the suspending agent is xanthan gum. The composition may include about 0.5 to 2.0% xanthan gum by weight, e.g., about 0.5 to 1.5% xanthan gum by weight, about 0.75 to 1.25% xanthan gum by weight. In some embodiments, the pharmaceutical composition includes about 1% xanthan gum by weight.

In some embodiments, formulations containing 0.5% xanthan gum by weight exhibit faster settling after dispersion in water than formulations containing greater amounts of xanthan gum. Formulations containing 2% xanthan gum by weight demonstrated roller sticking during roller compaction. Formulations containing 1% xanthan gum by weight, however, exhibited good processability and acceptable dispersion properties with minimal sedimentation and without any caking.

### Disintegrant

A pharmaceutical composition may include a disintegrant.

Examples of suitable disintegrants include sodium starch glycolate, crospovidone, cross-linked alginic acid, crosslinked starch, crosslinked alginate sodium, carmellose, carmellose calcium, croscarmellose sodium, glycerin fatty acid ester, low-substituted sodium carboxymethyl starch and partially pregelatinized starch.

The pharmaceutical composition may include about 0.5 to 1.5% disintegrant by weight e.g., about 0.75 to about 1.25% disintegrant by weight, about 0.75 to 1% disintegrant by weight. In one embodiment the disintegrant is croscarmellose sodium.

The pharmaceutical composition may include about 0.5 to 1.5% croscarmellose sodium by weight, e.g., about 0.75 to about 1.25% croscarmellose sodium by weight, about 0.75 to 1% croscarmellose sodium by weight. In some embodiments, the pharmaceutical composition includes about 1% croscarmellose sodium by weight.

In some embodiments, pharmaceutical compositions formed with croscarmellose sodium exhibit good processability and form uniform dispersions with water in under 30 seconds. Alternative formulations that lack croscarmellose sodium may require longer (e.g., about 1 minute) to disperse.

### Lubricant

A pharmaceutical composition may include a lubricant. Examples of suitable lubricants include, glyceryl behenate, glyceryl behaptate; sodium stearyl fumarate, stearic acid and salts thereof, including magnesium, calcium and sodium stearates; hydrogenated vegetable oils; silicon dioxide; talc; waxes; boric acid; sodium benzoate; sodium acetate; sodium fumarate; sodium chloride; DL-leucine; polyethylene glycol; sodium oleate; sodium lauryl sulfate; and magnesium lauryl sulfate.

The pharmaceutical composition may include about 0.4% to 0.8% lubricant by weight, e.g., about 0.5 to 0.7% lubricant by weight, about 0.5 to 0.6% lubricant by weight.

In one embodiment, the lubricant is magnesium stearate. The composition may include about 0.4% to 0.8% magnesium stearate by weight, e.g., about 0.5 to 0.7% magnesium stearate by weight, about 0.5 to 0.6% magnesium stearate by weight. In some embodiments, the composition includes about 0.6% magnesium stearate by weight.

### Glidant

A pharmaceutical composition may include a glidant. Examples of glidants include colloidal silicon dioxide, hydrated sodium sulfoaluminate, and talc.

The pharmaceutical composition may contain about 0.2% to 0.6% glidant by weight.

In one embodiment, the glidant is colloidal silicon dioxide. The pharmaceutical composition may contain about 0.2% to 0.6% colloidal silicon dioxide by weight. In some embodiments, the pharmaceutical composition includes about 0.4% colloidal silicon dioxide by weight.

### Sweetener

A pharmaceutical composition may include a sweetener. Examples of sweeteners include sucralose, sodium saccharin, aspartame, and neutrame.

The pharmaceutical composition may contain about 0.5% to 2.0% sweetener by weight, e.g., about 0.5 to 1.5% sucralose by weight, about 0.5 to 1.0% sucralose by weight.

In a one embodiment, the sweetener is sucralose.

The pharmaceutical composition may contain about 0.5% to 2.0% sucralose by weight, e.g., about 0.5 to 1.5% sucralose by weight, about 0.5 to 1.0% sucralose by weight. In some embodiments, the pharmaceutical composition includes about 1.0% sucralose by weight.

### Antioxidant

A pharmaceutical composition may include an antioxidant. Examples of antioxidants include 4-chloro-2,6-di-tert-butylphenol, tocopherol, alpha-tocopherol, alkylated diphenylamines, ascorbic acid, ascorbyl myristate, ascorbyl palmitate, ascorbyl stearate, beta-carotene, butylated hydroxyanisole, butylated hydroxytoluene, citric acid, cysteine, D-alpha-tocopheryl polyethylene glycol 1000 succinate, deferoxamine methanesulfonate, dodecyl gallate, ethylparaben, folic acid, fumaric acid, gallic acid, glutathione, lecithin, malic acid, methylparaben, monothioglycerol, N-acetyl cysteine, nordihydroguaiaretic acid, octyl gallate, p-phenylenediamine, potassium ascorbate, potassium metabisulfite, potassium sorbate, propionic acid, propyl gallate, retinol, sorbic acid, sodium ascorbate, sodium bisulfite, sodium hydrosulfite, sodium isoascorbate, sodium metabisulfite, sodium sulfite, sodium thiosulfate, tartaric acid, tert-butylhydroquinone, tocopheryl acetate, vitamin A, vitamin B6, vitamin B12, or vitamin E, or a combination thereof. In some embodiments of any of the foregoing pharmaceutical compositions, the antioxidant is ascorbic acid, tocopherol, retinol, sodium metabisulfite, ascorbyl palmitate, N-acetyl cysteine, glutathione, butylated hydroxytoluene, and/or butylated hydroxyanisole. In some embodiments of any of the foregoing pharmaceutical compositions, the antioxidant is ascorbic acid or sodium metabisulfite.

The pharmaceutical composition may contain about 0.05 to 0.2% antioxidant by weight, e.g., 0.05 to 0.15% antioxidant by weight, 0.08 to 0.1% antioxidant by weight.

In one embodiment, the antioxidant is sodium metabisulfite. The pharmaceutical composition may include may contain about 0.05 to 0.2% sodium metabisulfite by weight, e.g., about 0.05 to 0.15% sodium metabisulfite by weight, about 0.08 to 0.1% sodium metabisulfite by weight. In some embodiments, the pharmaceutical composition includes about 0.1% sodium metabisulfite by weight.

In other embodiments, the pharmaceutical composition does not include an antioxidant.

### Exemplary Composition

There are a wide variety of suitable formulations of the pharmaceutical composition of the present invention. Formulations suitable for oral administration include (a) powders, (b) capsules, tablets, lozenges, and troches, each containing a predetermined amount of the active ingredient, as solids or granules; (c) liquid solutions, such as an effective amount of the compound dissolved in diluents, such as water, saline, or juice; (d) suspensions in an appropriate liquid; and (e) suitable emulsions. Preferred are solid oral dosage forms such as capsule forms, tablet forms, and powder forms.

In some embodiments, a pharmaceutical composition of the invention is combined with a dosing vehicle prior to administration. In some embodiments of any of the foregoing compositions, the composition may be administered in a dosing vehicle with a viscosity of approximately 50-1750 centipoise (cP), e.g., to aid suspension and dosing of the pharmaceutical composition. One type of suspending agent that can be used is a combination of glycerin and sucrose in water (e.g., MEDISCA® oral mix with 2.5% glycerin and 27% sucrose in water).

In some embodiments of the invention, the composition is formulation B and is made up of sepiapterin and excipients in the ratios described in Table 9. In one embodiment, the composition contains 250 mg sepiapterin. In a second embodiment, the composition contains 1,000 mg sepiapterin.

TABLE 9
Formulation B

| | | Quantity (mg per Unit Dose) | |
|---|---|---|---|
| Component | % w/w | 250 mg Strength | 1000 mg Strength |
| Sepiapterin | 25.0 | 250.0 | 1000.0 |
| Microcrystalline | 21.0 | 210.0 | 840.0 |
| cellulose | | | |
| Isomalt | 40.0 | 400.0 | 1600.0 |
| Mannitol | 10.0 | 100.0 | 400.0 |
| Croscarmellose sodium | 1.0 | 10.0 | 40.0 |
| Xanthan gum | 1.0 | 10.0 | 40.0 |
| Colloidal silicon dioxide | 0.4 | 4.0 | 16.0 |
| Sucralose | 1.0 | 10.0 | 40.0 |
| Magnesium stearate | 0.6 | 6.0 | 24.0 |
| Total | 100 | 1000.0 | 4000.0 |

In a third and fourth embodiment, the composition is formulation C or D, and is made up of the ratios described in Table 10.

TABLE 10
Formulations C and D

| | Formulation C | Formulation D |
|---|---|---|
| Sepiapterin | 25 | 25 |
| Microcrystalline Cellulose | 20.9 | 41 |
| Isomalt | 40 | 30 |
| Mannitol | 10 | — |
| Croscarmellose Sodium | 1 | 1 |
| Xanthan gum | 1 | 1 |
| Colloidal Silicon Dioxide | 0.4 | 0.4 |
| Sodium Metabisulfite | 0.1 | — |
| Sucralose | 1 | 1 |
| Magnesium Stearate | 0.6 | 0.6 |
| Total | 100 | 100 |

### Methods of Preparing Formulation

In some embodiments, the pharmaceutical compositions of the invention are prepared via dry granulation. In some embodiments of the instant invention, the dry granulation process may include roller compaction, and subsequent milling, blending, and sachet filling. Thus, the selection of excipients that are compatible with the use of a roller compactor, such as excipients that do not cause roller sticking, is important.

One process for making the composition of the invention employs roller compaction according to the following steps: (1) sieving sepiapterin and at least one water-soluble diluent, water-insoluble diluent, disintegrant, suspending agent, glidant, and sweetener, and blending, forming a pre-blended mixture, (2) sieving a lubricant, (3) mixing the lubricant with the preblended mixture to form a lubricated preblended mixture, (4) preparing ribbons of the lubricated preblended mixture using a roller compactor, (5) milling the ribbons to form a milled formulation, (6) blending the milled formulation with a blender, forming the pharmaceutical composition. The process for making the composition of the invention may additionally include filling the pharmaceutical composition into sachets.

### Method of Use

The pharmaceutical compositions of sepiapterin of the instant invention may serve to increase intracellular BH₄ levels in a subject. Thus, the pharmaceutical compositions of sepiapterin disclosed herein may serve as a useful therapeutic for diseases associated with low intracellular BH₄ levels or with dysfunction of various BH₄ dependent metabolic pathways including, but not limited to, primary tetrahydrobiopterin deficiency, GTPCH deficiency, gastroparesis, hyperphenylalaninemia, 6-pyruvoyl-tetrahydropterin synthase (PTPS) deficiency, DHPR deficiency, sepiapterin reductase deficiency, dopamine responsive dystonia, Segawa Syndrome, tyrosine hydroxylase deficiency, phenylketonuria, DNAJC12 deficiency, Parkinson's Disease, depression due to Parkinson's Disease, impulsivity in Parkinson's patients, major depression, Autism spectrum, ADHD, schizophrenia, Bipolar disorder, cerebral ischemia, restless leg syndrome, obsessive compulsive disorder, anxiety, aggression in Alzheimer's disease, cerebrovascular disorders, spasm after subarachnoidal hemorrhage, myocarditis, coronary vasospasm, cardiac hypertrophy, arteriosclerosis, hypertension, thrombosis, infections, endotoxin shock, hepatic cirrhosis, hypertrophic pyloric stenosis, gastric mucosal injury, pulmonary hypertension, renal dysfunction, impotence, and hypoglycemia. Thus, the pharmaceutical compositions of sepiapterin, in accordance with the present invention can be administered to a patient in an effective amount to obtain a treatment or amelioration of the disease, disorder or condition.

### Dosage

Sepiapterin can be used in any suitable dose. Suitable doses and dosage regimens can be determined by conventional range finding techniques. Generally, treatment is initiated with smaller dosages, which are less than the optimum dose. Thereafter, the dosage is increased by small increments until optimum effect under the circumstances is reached. For convenience, the total daily dosage may be divided and administered in portions during the day if desired. In proper doses and with suitable administration of certain compounds, the present invention provides for a wide range of responses.

In some embodiments, the dose is an amount sufficient to produce levels of BH₄ in the CNS, e.g., as measured in the CSF and/or sufficient to produce a therapeutic result, e.g., increased levels of serotonin or dopamine in the CNS. In some embodiments, the dose is an amount sufficient to produce levels of BH₄ in plasma.

In one aspect the pharmaceutical composition of sepiapterin is provided as an oral powder in a sachet. The sachet can be of any suitable size. In some embodiments, the pharmaceutical composition of sepiapterin is provided as 250 mg or 1000 mg sachets. The calculated daily dose based on body weight may be rounded to the nearest multiple of 250 mg or 1000 mg, as appropriate. For instance, a calculated dose of 1251 to 1374 mg may be rounded down to 1250 mg corresponding to 1×250 mg sachet and 1×1000 mg sachet. A calculated dose of 1375 to 1499 mg may be rounded up to 1500 mg corresponding to 2×250 mg sachets and 1×1000 mg sachet.

The full contents of a single 250 mg or 1000 mg sachet may be suspended in 10 mL or 20 ml of water or fruit juice, respectively. Once added, the combination may be shaken or stirred for at least 30 seconds to form the suspension. After mixture, all of the resulting suspension or an appropriate quantity of the resulting suspension depending on the weight of the individual may be measured and administered PO to the subject immediately.

For Subjects ≥2 Years of Age Weighing ≤18 kg Administering 20 mg/kg Dose

For subjects ≥2 years of age weighing ≤18 kg, the pharmaceutical composition of sepiapterin may be suspended in 9 mL of water or apple juice per 250 mg sachet and an aliquot of this suspension corresponding to a 20 mg/kg dose may be administered orally via an oral dosing syringe. Table 1 provides dosing information for subjects at 20 mg/kg doses. A graduated dosing syringe can be used for subjects weighing ≤18 kg for this dose.

TABLE 11
20 mg/kg/day Dosing Table for Subjects ≥2 Years
of Age Weighing ≤18 kg

| | Target Dose: 20 mg/kg/day^{b} | | | |
|---|---|---|---|---|
| | | Pharmaceutical | | |
| Subject | | Composition of | Dilution | Administered |
| Weight | Dose | Sepiapterin, 250 mg Sachets | Volume | Dose Volume |
| (kg)^{a} | (mg) | Suspended^{c} | (mL)^{d} | (mL)^{e} |
| 8 | 160 | 1 | 9 | 6.4 |
| 9 | 180 | 1 | 9 | 7.2 |
| 10 | 200 | 1 | 9 | 8 |
| 12 | 240 | 1 | 9 | 9.6 |
| 12.5 | 250 | 1 | 9 | 10 |
| 15 | 300 | 2 | 18 | 12 |
| 18 | 360 | 2 | 18 | 14.4 |

^{a}Round the subject's weight to the nearest listed weight in the table in kg. For instance, a subject who weighs between 8.01 and 8.49 kg may be rounded down to 8 kg and a subject who weighs between 8.7550 and 8.99 kg may be rounded up to 9 kg.
^{b}Target dose of 20 mg/kg/day for subjects.
^{c}Pharmaceutical composition of sepiapterin, 250 mg provided in single use sachets.
^{d}Volume of liquid (water or apple juice) to suspend the pharmaceutical composition of sepiapterin, 250 mg.
^{e}Discard remainder of mixture after volume to be administered is drawn.

For Subjects ≥2 Years of Age Weighing ≤15 kg Administering 40 and 60 mg/kg Doses

For subjects ≥2 years of age weighing ≤15 kg, the pharmaceutical composition of sepiapterin may be suspended in 9 mL of water or apple juice per 250 mg sachet and an aliquot of this suspension corresponding to a 40 and 60 mg/kg dose may be administered orally via an oral dosing syringe. Table 12 and Table 13 provide dosing information for subjects at 40 and 60 mg/kg doses, respectively. A graduated dosing syringe can be used for subjects weighing ≤15 kg for these 2 doses.

TABLE 12
40 mg/kg/day Dosing Table for Subjects ≥2 Years
of Age Weighing ≤15 kg

| | Target Dose: 40 mg/kg/day^{b} | | | |
|---|---|---|---|---|
| | | Pharmaceutical | | |
| Subject | | Composition of | Dilution | Administered |
| Weight | Dose | Sepiapterin, 250 mg Sachets | Volume | Dose Volume |
| (kg)^{a} | (mg) | Suspended^{c} | (mL)^{d} | (mL)^{e} |
| 8 | 320 | 2 | 18 | 12.8 |
| 9 | 360 | 2 | 18 | 14.4 |
| 10 | 400 | 2 | 18 | 16 |
| 12 | 480 | 2 | 18 | 19.2 |
| 12.5 | 500 | 2 | 18 | 20 |
| 15 | 600 | 3 | 27 | 24 |

^{a}Round the subject's weight to the nearest listed weight in the table in kg. For instance, a subject who weighs between 8.01 and 8.49 kg may be rounded down to 8 kg and a subject who weighs between 8.50 and 8.99 kg may be rounded up to 9 kg.
^{b}Target dose of 40 mg/kg/day for subjects.
^{c}Pharmaceutical composition of sepiapterin, 250 mg provided in single use sachets.
^{d}Volume of liquid (water or apple juice) to suspend the pharmaceutical composition of sepiapterin, 250 mg.
^{e}Discard remainder of mixture after volume to be administered is drawn.

TABLE 13
60 mg/kg/day Dosing Table for Subjects ≥2 Years
of Age Weighing ≤15 kg

| | Target Dose: 60 mg/kg/day^{b} | | | |
|---|---|---|---|---|
| | | Pharmaceutical | | |
| Subject | | Composition of | Dilution | Administered |
| Weight | Dose | Sepiapterin, 250 mg Sachets | Volume | Dose Volume |
| (kg)^{a} | (mg) | Suspended^{c,f} | (mL)^{d} | (mL)^{e} |
| 8 | 480 | 2 | 18 | 19.2 |
| 9 | 540 | 3 | 27 | 21.6 |
| 10 | 600 | 3 | 27 | 24 |
| 12 | 720 | 3 | 27 | 28.8 |
| 12.5 | 750 | 3 | 27 | 30 |
| 15 | 900 | 1 × 1000 mg sachet | 36 | 36 |

^{a}Round the subject's weight to the nearest listed weight in the table in kg. For instance, a subject who weighs between 8.01 and 8.49 kg may be rounded down to 8 kg and a subject who weighs between 8.75 and 8.99 kg may be rounded up to 9 kg.
^{b}Target dose of 60 mg/kg/day for subjects.
^{c}Pharmaceutical composition of sepiapterin, 250 mg provided in single use sachets.
^{d}Volume of liquid (water or apple juice) to suspend the pharmaceutical composition of sepiapterin, 250 mg.
^{e}Discard remainder of mixture after volume to be administered is drawn.
^{f}Use 1000 mg sachets when indicated.

### For Subjects <6 Months of Age Weighing ≤12 kg Administering 7.5 Ma/Kg Dose

For subjects <6 months of age weighing $12 kg, the pharmaceutical composition of sepiapterin may be suspended in 9 mL of water or apple juice per 250 mg sachet and an aliquot of this suspension corresponding to a 7.5 mg/kg dose may be administered orally via an oral dosing syringe. Table 14 provides dosing information for subjects <6 months at 7.5 mg/kg dose. A graduated dosing syringe will be provided for subjects weighing ≤12 kg for this dose.

TABLE 14
7.5 mg/kg/day Per Day Dosing Table for
Subjects <6 Months of Age Weighing ≤12 kg

| | Target Dose: 7.5 mg/kg/day^{b} | | | |
|---|---|---|---|---|
| | | Pharmaceutical | | |
| Subject | | Composition of | Dilution | Administered |
| Weight | Dose | Sepiapterin, 250 mg Sachets | Volume | Dose Volume |
| (kg)^{a} | (mg) | Suspended^{c} | (mL)^{d} | (mL)^{e} |
| 1 | 7.5 | 1 | 9 | 0.3 |
| 1.2 | 9 | 1 | 9 | 0.36 |
| 1.4 | 10.5 | 1 | 9 | 0.42 |
| 1.7 | 12.8 | 1 | 9 | 0.51 |
| 2 | 15 | 1 | 9 | 0.6 |
| 2.5 | 18.8 | 1 | 9 | 0.75 |
| 3 | 22.5 | 1 | 9 | 0.9 |
| 3.5 | 26.3 | 1 | 9 | 1.05 |
| 4 | 30 | 1 | 9 | 1.2 |
| 4.5 | 33.8 | 1 | 9 | 1.35 |
| 5 | 37.5 | 1 | 9 | 1.5 |
| 6 | 45 | 1 | 9 | 1.8 |
| 7 | 52.5 | 1 | 9 | 2.1 |
| 8 | 60 | 1 | 9 | 2.4 |
| 9 | 67.5 | 1 | 9 | 2.7 |
| 10 | 75 | 1 | 9 | 3 |
| 12 | 90 | 1 | 9 | 3.6 |

^{a}Round the subject's weight to the nearest listed weight in the table in kg. For instance, a subject who weighs between 2.01 and 2.24 kg may be rounded down to 2 kg and a subject who weighs between 4.75 and 4.99 kg may be rounded up to 5 kg.
^{b}Target dose of 7.5 mg/kg/day for subjects
^{c}Pharmaceutical composition of sepiapterin, 250 mg provided in single use sachets.
^{d}Volume of liquid (water or apple juice) to suspend the pharmaceutical composition of sepiapterin, 250 mg.
^{e}Discard remainder of mixture after volume to be administered is drawn.

For Subjects ≥6 to <12 Months of Age Weighing ≤15 kg Administering 15 mg/kg Dose

For subjects ≥6 to <12 months of age weighing ≤15 kg, the pharmaceutical composition of sepiapterin may be suspended in 9 mL of water or apple juice per 250 mg sachet and an aliquot of this suspension corresponding to a 15 mg/kg dose may be administered orally via an oral dosing syringe. Table provides dosing information for subjects ≥6 to <12 months of age at 15 mg/kg dose. A graduated dosing syringe will be provided for subjects weighing ≤15 kg for this dose.

TABLE 15
15 mg/kg/day Dosing Table for Subjects ≥6
to <12 Months of Age Weighing ≤15 kg

| | Target Dose: 15 mg/kg/day^{b} | | | |
|---|---|---|---|---|
| | | Pharmaceutical | | |
| Subject | | Composition of | Dilution | Administered |
| Weight | Dose | Sepiapterin, 250 mg Sachets | Volume | Dose Volume |
| (kg) ^{a} | (mg) | Suspended^{c} | (mL)^{d} | (mL)^{e} |
| 4 | 60 | 1 | 9 | 2.4 |
| 4.5 | 67.5 | 1 | 9 | 2.7 |
| 5 | 75 | 1 | 9 | 3 |
| 6 | 90 | 1 | 9 | 3.6 |
| 7 | 105 | 1 | 9 | 4.2 |
| 8 | 120 | 1 | 9 | 4.8 |
| 9 | 135 | 1 | 9 | 5.4 |
| 10 | 150 | 1 | 9 | 6 |
| 12 | 180 | 1 | 9 | 7.2 |
| 15 | 225 | 1 | 9 | 9 |

^{a} Round the subject's weight to the nearest listed weight in the table in kg. For instance, a subject who weighs between 4.01 and 4.24 kg may be rounded down to 4 kg and a subject who weighs between 4.75 and 4.99 kg may be rounded up to 5 kg.
^{b}Target dose of 15 mg/kg/day for subjects ≥6 months but <12 months.
^{c}Pharmaceutical composition of sepiapterin, 250 mg provided in single use sachets.
^{d}Volume of liquid (water or apple juice) to suspend the pharmaceutical composition of sepiapterin, 250 mg.
^{e}Discard remainder of mixture after volume to be administered is drawn.

For Subjects ≥12 Months to <2 Years of Age Weighing ≤18 kg Administering 30 mg/kg Dose

For subjects ≥12 months to <2 years of age weighing ≤18 kg, the pharmaceutical composition of sepiapterin may be suspended in 9 mL of water or apple juice per 250 mg sachet and an aliquot of this suspension corresponding to a 30 mg/kg dose may be administered orally via an oral dosing syringe. Table provides dosing information for subjects ≥12 months to <2 years of age at 30 mg/kg dose. A graduated dosing syringe will be provided for subjects weighing ≤18 kg for this dose.

TABLE 16
30 mg/kg/day Dosing Table for Subjects ≥12 Months
to <2 Years of Age Weighing ≤18 kg

| | Target Dose: 30 mg/kg/day^{b} | | | |
|---|---|---|---|---|
| | | Pharmaceutical | | |
| Subject | | Composition of | Dilution | Administered |
| Weight | Dose | Sepiapterin, 250 mg Sachets | Volume | Dose Volume |
| (kg)^{a} | (mg) | Suspended^{c} | (mL)^{d} | (mL)^{e} |
| 5 | 150 | 1 | 9 | 6 |
| 6 | 180 | 1 | 9 | 7.2 |
| 7 | 210 | 1 | 9 | 8.4 |
| 8 | 240 | 1 | 9 | 9.6 |
| 9 | 270 | 2 | 18 | 10.8 |
| 10 | 300 | 2 | 18 | 12 |
| 12 | 360 | 2 | 18 | 14.4 |
| 15 | 450 | 2 | 18 | 18 |
| 18 | 540 | 3 | 27 | 21.6 |

^{a}Round the subject's weight to the nearest listed weight in the table in kg. For instance, a subject who weighs between 5.01 and 5.49 kg may be rounded down to 5 kg and a subject who weighs between 5.50 and 5.99 kg may be rounded up to 6 kg.
^{b}Target dose of 30 mg/kg/day for subjects.
^{c}Pharmaceutical composition of sepiapterin, 250 mg provided in single use sachets.
^{d}Volume of liquid (water or apple juice) to suspend the pharmaceutical composition of sepiapterin, 250 mg.

In some embodiments, the pharmaceutical composition of the invention is a yellow to orange powder that is packaged in aluminum sachets containing either 250 mg or 1000 mg of sepiapterin and may be stored refrigerated at 2° C. to 8° C. Constituted sepiapterin suspension may be administered immediately. During the time from constitution to administration, the resulting suspension may be stored at room temperature. The full contents of a single 250 mg or 1000 mg sachet of the composition may be suspended in 10 mL or 20 mL of water or fruit juice, respectively. Once added, the combination may be shaken or stirred for at least 30 seconds to form the suspension. After preparation, all of the resulting suspension or an appropriate quantity of the resulting suspension depending on the weight of the individual may be measured and administered PO to the subject immediately.

### EXAMPLES

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention. As such, the following examples are provided to teach various aspects of the present invention. Except where otherwise noted below, these examples represent individual embodiments of the aspects of this invention and one skilled in the art will recognize that additional examples can be generated in order to equally teach the aspects of the present invention.

The following examples include illustrations of aspects of the invention. The sepiapterin used in the formulations illustrated in all of the examples below was in the form of crystalline Form F. The examples are not to be construed as limitations

### Example 1—Formulation A

The following formulation of sepiapterin, Formulation A, was previously used in Phase I clinical trial studies of phenylketonuria by Censa Pharmaceuticals (now, PTC Therapeutics, Inc.). That same formulation was used as a reference formulation in the studies described in Examples 8-10 and 12, below. Formula A is an oral powder formulation having the following composition: sepiapterin (23%), ascorbic acid (5%), microcrystalline cellulose (57%), croscarmellose sodium (9%), and colloidal silicon dioxide (6%), where all percentages are percent by weight. A method of making that formulation is disclosed in Example 1 of the international application published as WO 2019/046849, incorporated by reference herein.

A summary of the Phase I clinical trials where that formulation was used can be found in Smith et al, “Phase I clinical evaluation of CNSA-001 (sepiapterin), a novel pharmacological treatment for phenylketonuria and tetrahydrobiopterin deficiencies in healthy volunteers,” Molecular Genetics and Metabolism 126 (2019) 406-412. A description of that study can also be found on clinicaltrials.gov.

### Example 2. Drug Excipient Compatibility (DEC) Study Using Accelerated Stability Assessment Program (ASAP)

Several excipients were screened to develop a sepiapterin formulation to improve processability, stability and palatability:
Binary mixtures of drug substance and different diluents (isomalt, microcrystalline cellulose, mannitol) at a 1:4 w/w ratio (drug substance:diluent).
Ternary mixture of drug substance, diluent, and other functional excipients such as a lubricant (magnesium stearate), disintegrants (croscarmellose sodium, sodium starch glycolate), a glidant (colloidal silicon dioxide), a binder (Polyvinyl pyrrolidone K30), an acidifier (citric acid), a surfactant (Poloxamer 407), suspending agents (hydroxyethyl cellulose, xanthan gum) and a sweetener (sucralose) at a 1:3.75:0.25 w/w ratio (drug substance:diluent:other excipients).

The samples of binary and ternary mixtures were stored at 40° C., 40° C./75% RH, 60° C., 60° C./75% RH, 70° C. and 70° C./50% RH for 2 weeks using accelerated stability assessment protocol. Stress conditions were selected for the DEC study to observe potential degradation kinetics in a shorter duration. Any change in the assay and degradant levels were assessed for stressed samples as an incompatibility indicator and compared against the unstressed control samples.

No significant change in the assay and degradant levels were observed for both binary and ternary mixtures at the stressed storage conditions except the mixtures containing citric acid and Poloxamer 407 where the degradation products increased from 0.61 to 3.59% w/w and from 0.47 to 2.06% w/w, respectively after 14 days of exposure at 40° C./75% RH.

### Blend Stability

Further, to evaluate any potential interactions of sepiapterin with excipient mixtures, a prototype blend stability study was conducted. The prepared blends were stored at 50° C./75% RH, 60° C., 60° C./75% RH, 70° C. and 70° C./50% RH for 2 weeks. As sepiapterin has the propensity to undergo oxidative degradation, some selected antioxidants have been evaluated. Different blends of sepiapterin and excipients were prepared to evaluate the impact of drug loading, diluent ratios, and different antioxidants (sodium metabisulfite, propyl gallate, butylated hydroxytoluene) on assay and degradation products.

The blend stability study demonstrated that the blend containing 39% w/w of microcrystalline cellulose showed slightly higher total degradation products (2.5% w/w) after 2 weeks of exposure at 60° C./75% RH as compared to the blend containing 19% w/w of microcrystalline cellulose which had total degradation products of 2.0% w/w.

The blend containing 25% w/w to 50% w/w of sepiapterin did not show a considerable impact on the assay and degradation products. Therefore, 25% w/w to 50% w/w drug loading levels may be employed.

Antioxidant Evaluation: The presence of antioxidants in the blends did not demonstrate a considerable improvement in blend stability as indicated by the total degradation products following 2 weeks of exposure at 60° C./75% RH. The amount of degradation products for a control blend (without antioxidant) and the blends containing sodium metabisulfite, butylated hydroxytoluene, and propyl gallate, was 2.14, 1.83, 2.01, and 2.31% w/w, respectively.

### Example 3. Manufacturing of Formulations

Formulations were manufactured at 100 g scale by roller compaction using the compositions as described in Table 17. The formulations were designed to evaluate the effect of formulation variables including drug loading, ratios of diluents and levels of other excipients on the processability, as shown in Table 17. Process observations and in-process data are shown in Table 18.

TABLE 17
Prototypes formulation for processability
and optimization of excipient levels

| | Batch | Batch | Batch | Batch | Batch | Batch |
|---|---|---|---|---|---|---|
| | 1 | 2 | 3 | 4 | 5 | 6 |
| Sepiapterin | 25 | 25 | 25 | 25 | 25 | 25 |
| MCC (PH112) | 40 | 20 | 20 | 21 | 21 | 41 |
| Galeniq 720 | 15 | 25 | 40 | 30 | 40 | 30 |
| (Isomalt) | | | | | | |
| Pearlitol DC 300 | 15 | 25 | 10 | 20 | 10 | 0 |
| Croscarmellose | 1 | 1 | 1 | 1 | 1 | 1 |
| Sodium | | | | | | |
| Sodium Starch | 1 | 1 | 1 | 1 | 1 | 1 |
| Glycolate | | | | | | |
| Xanthan gum | 1 | 1 | 1 | 0.4 | 0.4 | 0.4 |
| Colloidal Silicon | 0.4 | 0.4 | 0.4 | 1 | 1 | 1 |
| dioxide | | | | | | |
| Sucralose | 1 | 1 | 1 | 0.6 | 0.6 | 0.6 |
| Magnesium | 0.6 | 0.6 | 0.6 | 0.6 | 0.6 | 0.6 |
| Stearate | | | | | | |
| Total | 100 | 100 | 100 | 100 | 100 | 100 |

TABLE 18
Processability observation and in-process data for formulations

| | Preblend | | | | | | | | | | | |
|---|---|---|---|---|---|---|---|---|---|---|---|---|
| | Flow from | | | | | | Bulk granule attributes | | | | | |
| | Hopper | Ribbon attributes | | | | | | | | | | Dispersion in |
| Batch | Screw | Roll | | | | DT* | BD | TD | | Carr | | water Time (s)/ |
| # | speed ~30-40 | Stickiness | Appearance | Thickness | Hardness | (min) | g/cc | g/cc | HR | Index | Flow** | Appearance |
| 1 | Good | No | Continuous | 0.8 | +++ | <1 | 0.57 | 0.695 | 1.22 | 21.9 | Fair/ | <30 s/ |
| | | | | | | | | | | | Passable | Uniform |
| | | | | | | | | | | | | dispersion |
| 2 | Good | No | Continuous | 0.8 | ++ | <1 | 0.55 | 0.67 | 1.22 | 21.8 | Fair/ | <30 s/ |
| | | | | | | | | | | | Passable | Uniform |
| | | | | | | | | | | | | dispersion |
| 3 | Good | No | Continuous | 0.8 | ++++ | ~1 | 0.56 | 0.7 | 1.25 | 25.0 | Fair/ | <30 s/ |
| | | | | | | | | | | | Passable | Uniform |
| | | | | | | | | | | | | dispersion |
| 4 | Good | No | Continuous | 0.8 | +++ | <1 | 0.56 | 0.67 | 1.20 | 19.6 | Fair/ | <30 s |
| | | | | | | | | | | | Passable | Uniform |
| | | | | | | | | | | | | dispersion |
| 5 | Good | No | Continuous | 0.8 | ++++ | ~1 | 0.56 | 0.69 | 1.23 | 23.2 | Fair/ | <30 s |
| | | | | | | | | | | | Passable | Uniform |
| | | | | | | | | | | | | dispersion |
| 6 | Good | No | Continuous | 0.8 | ++++ | <1 | 0.56 | 0.7 | 1.25 | 25.0 | Fair/ | <30 s |
| | | | | | | | | | | | Passable | Uniform |
| | | | | | | | | | | | | dispersion |

In the tables above, BD refers to Bulk Density, DT refers to Disintegration time, HR refers to Hausner ratio and TD refers to tapped density. Disintegration time is for a 0.5-inch ribbon. The plus signs in the table refer to the following observed characteristics: “+” is soft, “++” is medium hard, “+++” is hard, and “++++” is very hard.

### Example 4. Drug Loading, Excipients, and their Respective Levels

### Drug Loading

During the roller compaction process, roller sticking was observed for formulations containing 40% w/w or higher drug loading. No roller sticking was observed for the formulations containing 25% w/w of sepiapterin.

### Diluents and Levels

The pharmaceutical composition of sepiapterin may be intended to disperse in aqueous media prior to oral administration. MCC is a water insoluble excipient and could influence the sedimentation properties after the reconstitution of the pharmaceutical composition of sepiapterin in liquid vehicles. Therefore, to enhance the sedimentation properties, dose recovery, and mouthfeel after reconstitution, lower levels of microcrystalline cellulose (20-40%) and two additional water-soluble diluents (i.e., isomalt and mannitol) were employed. The 20-40% w/w level of microcrystalline cellulose provided the best processability, ribbon characteristics, and in-process results (Table 18).

The formulations with lower amounts of mannitol (0-10% w/w) did not adhere to rollers and provided continuous ribbons.

The formulations containing 30-40% w/w level of isomalt provided good processability characteristics (Table 18).

Overall, mannitol (0-10%), isomalt (30-40%), and microcrystalline cellulose (20-40%) may be employed.

### Disintegrant Level

Croscarmellose sodium is added to the drug product to facilitate disintegration of granules upon constitution in aqueous media. The formulation batches manufactured with 1% w/w croscarmellose sodium exhibited good processibility and formed uniform dispersions with water (5, <30 seconds) as compared to a reference R2 formulation without any croscarmellose sodium (R1, ˜1 min dispersion time).

### Glidant Level

A reference formulation R2 and 5, with two different levels of colloidal silicon dioxide (i.e., 0.2% and 0.4% w/w for respectively) were manufactured. Unlike 5, the preblend as well as the milled granules of R2 showed poor flow properties, which could potentially impact the weight uniformity during the sachet filling operation.

### Lubricant Level

Three formulations containing 0.4%, 0.6%, and 0.8% w/w magnesium stearate, respectively were evaluated. Roll sticking was observed for prototype PTC 30 with 0.4% w/w magnesium stearate. The formulation with 0.6% w/w magnesium stearate yielded acceptable ribbons without roll sticking.

### Suspending Agent and Level

Hydroxyethyl cellulose and xanthan gum were evaluated as suspending agents at a 2% w/w level to form uniform dispersions upon constitution in water. Unlike xanthan gum, the hydroxyethyl cellulose containing formulation resulted in flocculated suspension with rapid sedimentation after constitution with water. Three different levels of xanthan gum, 0.5, 1, and 2% w/w, were evaluated. The formulation containing 0.5% w/w xanthan gum exhibited faster settling after constitution in water, whereas the formulation containing 2% w/w xanthan gum demonstrated roller sticking. Moreover, the 1.0% w/w xanthan gum formulation exhibited good processibility as well as acceptable dispersion properties with minimal sedimentation without any caking.

### Antioxidant and Level

The effects of two antioxidants, sodium metabisulphite and a 1:1 mixture of BHT (butylated hydroxytoluene):BHA (butylated hydroxyanisole) were evaluated after two weeks of storage at 60° C./75% RH. Sodium metabisulphite was evaluated at levels of 0.05% w/w, 0.1% w/w, and 0.2% w/w. The 1:1 BHT:BHA mixture was evaluated at levels of 0.01% w/w, 0.05% w/w, and 0.1% w/w.

No significant change in the assay and total degradation products was observed for all formulations containing antioxidants as compared to the control formulation without any antioxidants.

### Example 5. Manufacturing Process

Unformulated sepiapterin has low bulk density and exhibits poor flow characteristics. To improve the flow properties of the sepiapterin composition of the invention, a dry granulation approach was selected. The manufacturing process included preblending, roller compaction followed by milling, final blending, and sachet filling. The batch analyses data from the selected manufacturing process yielded acceptable drug product within the specification limit. The representative manufacturing process includes.
1) sieving sepiapterin and at least one water-soluble diluent, water-insoluble diluent, disintegrant, suspending agent, glidant, and sweetener, and blending, forming a pre-blended mixture;
2) sieving a lubricant;
3) mixing the lubricant with the preblended mixture to form a lubricated preblended mixture;
4) preparing ribbons of the lubricated preblended mixture using a roller compactor;
5) milling the ribbons to form a milled formulation;
6) blending the milled formulation with a blender, forming the pharmaceutical composition; and optionally
7) filling the pharmaceutical composition into sachets using a sachet filler.

### Example 6. Hygroscopicity of Sepiapterin Drug Substance and Drug Product

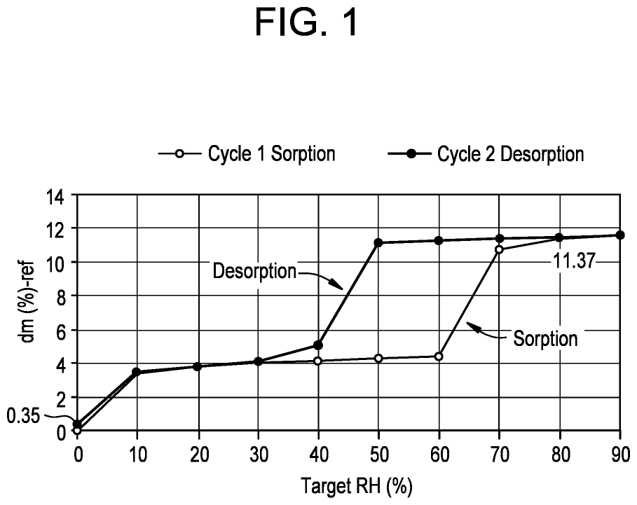

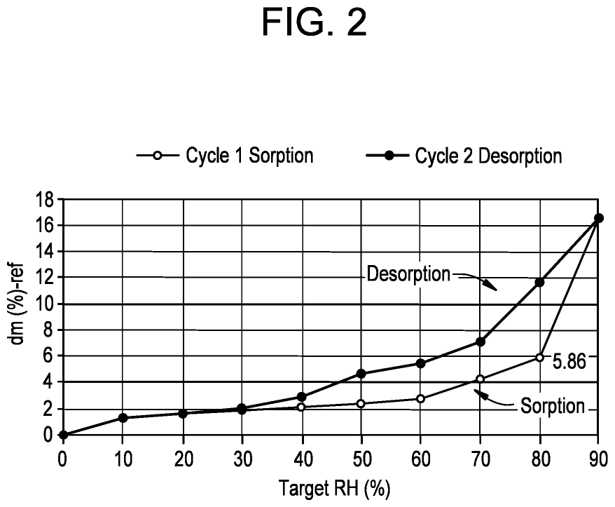

To understand optimum humidity conditions in the processing area, the hygroscopicity of sepiapterin and the pharmaceutical composition described in Table 17 was evaluated using dynamic vapor sorption (DVS). The sorption/desorption plots are illustrated in FIGS. 1 and 2.

This material is moderately-hygroscopic (2-15% w/w weight gain at 80% RH), and between 0 and 0.35% of adsorbed water remains after desorption. Therefore, moisture protection procedures are implemented during storage, handling, processing, packaging, and stability of the material. Based on DVS data and hygroscopicity, the drug product manufacturing area needs to be maintained at or below 50% RH conditions.

### Example 7. Stability Assessment of Lead Prototype Formulations

Based on the above data, three formulations were selected for further stability assessment (Table 19).

TABLE 19
Lead prototype formulations

| | Formulation B | Formulation C | Formulation D |
|---|---|---|---|
| Variation | No antioxidant | Antioxidant 1 | Highest level |
| | | | of Avicel |
| | | | No antioxidant |
| Sepiapterin | 25 | 25 | 25 |
| Microcrystalline | 21 | 20.9 | 41 |
| Cellulose | | | |
| (Avicel PH112) | | | |
| Isomalt | 40 | 40 | 30 |
| (Galeniq 720) | | | |
| Mannitol | 10 | 10 | — |
| (Pearlitol DC 300) | | | |
| Croscarmellose | 1 | 1 | 1 |
| Sodium | | | |
| Xanthan gum | 1 | 1 | 1 |
| Colloidal | 0.4 | 0.4 | 0.4 |
| Silicon Dioxide | | | |
| Sodium | — | 0.1 | — |
| Metabisulfite | | | |
| Sucralose | 1 | 1 | 1 |
| Magnesium | 0.6 | 0.6 | 0.6 |
| Stearate | | | |
| Total | 100 | 100 | 100 |

The stability assessment of the three sepiapterin formulations (formulation A, formulation B, formulation C) was made using two approaches: (1) an Accelerated Stability Assessment Protocol (ASAP) study to predict the long-term stability of each formulation, and (2) a stability assessment at ICH conditions

### Accelerated Stability Assessment Protocol Study

The experimental design and data analysis from a short-term (64 days) ASAP study was used to predict the long-term stability performance of sepiapterin formulations. The projected shelf life of the formulations was studied using a seven-point protocol designed to model relative humidity and temperature effects in an eight-week timeframe. Samples were stored at the designated stress conditions and analyzed at timepoints as shown in Table 20. The samples were analyzed for assay and degradants using an HPLC-UV method.

TABLE 20
ASAP Study Protocol

| Conditions | | 1^{st} Pull | 2^{nd} Pull | 3^{rd} Pull | 4^{th} Pull |
|---|---|---|---|---|---|
| T (° C.) | % RH | (days) | (days) | (days) | (days) |
| 50 | 54 | 7 | 15 | 32 | 64 |
| 50 | 73 | 7 | 15 | 32 | 64 |
| 60 | 5 | 7 | 15 | 32 | 64 |
| 60 | 50 | 7 | 15 | 32 | 64 |
| 60 | 75 | 7 | 15 | 32 | 64 |
| 70 | 9 | 7 | 15 | 32 | 64 |
| 70 | 34 | 7 | 15 | 32 | 64 |

Reaction rates follow the humidity-modified Arrhenius kinetics for the assay and most of the impurities generated by drug degradation. Arrhenius kinetics is a linear dependence of the natural logarithm of the reaction rate k, versus the % RH and the reciprocal of the absolute temperature T. This enables the modeling of both relative humidity and temperature to provide a rapid determination of the fitted parameters (In A, Ea, and B) for the humidity corrected Arrhenius equation (Eq. 1)

$$ln k = ln A - \frac{{E}_{a}}{R} * \frac{1}{T} + B * \% RH \qquad Eq . 1$$

where:
k=growth rate of degradant from control expressed as %/day
In A=constant, determined by fit
Ea=Energy of Activation
R=Universal gas constant
Ea/R=constant, determined by the fit (Arrhenius model)
T=temperature in ° Kelvin
B=constant, determined by fit

A multilinear regression approach was used to estimate the Ln A, Ea, and B terms from the seven accelerated storage conditions. The degradation levels in the formulations packaged in sealed aluminum foil sachets were predicted using input parameters such as the packaging moisture vapor transmission rate (MVTR), the sample water activity, and the moisture isotherm profile associated with the drug formulations.

This information together with the Ln A, Ea, and B values were used to predict the levels of drug product degradation in the packaging at long term and accelerated storage conditions.

The parameters given in Table 21 were utilized in determining ASAP predictions for sepiapterin formulations, wherein RRT refers to the relative retention time of the degradant.

TABLE 21
Parameters Utilized in ASAP Predictions

| Software | ASAP prime Version 6.01 |
|---|---|
| Target Degradants xq1′′′′ | RRT 0.52 |
| | RRT 0.61 |
| | RRT 0.66 |
| | RRT 0.74 |
| | RRT 0.88 |
| | RRT 0.95 |
| External Conditions | 5° C. (2° C.-8° C.) / 25° C./60% RH |
| Packaging Configuration | Foil Sachet Packaging |
| | 0.013 g/m2/day (23° C./75%)-estimated |
| | 0.05 g/m2/day (38° C./90%) |
| Weight per sample (mg) | 1000 mg |

The experimental data were fitted into an ASAP model to predict degradant formation over the expected life span of the formulation. The ASAP predictions have lower and upper 95% confidence intervals. The upper bound confidence interval represents the worst-case scenario for the product's use-by period. This means that the probability of meeting the proposed acceptance criteria is significantly reduced when the upper bound confidence interval reaches the specification limit before the assigned expiry date.

A short-term (i.e., 64 days) accelerated stability protocol suitable for the simulation of the long-term stability performance of Sepiapterin Powder for Oral Use formulations packaged in aluminum foil sachets configuration was conducted. The shelf-life predictions were based at storage conditions of 5° C. (Table 22) and 25° C./60% RH (Table 23).

### Example 8—Study of Relative Bioavailability of Three Formulations

### Formulation

TABLE 22
Prediction of Degradant Profile at 5° C. Conditions for Storage up to 36 Months

| | | | Formulation B | | | Formulation C | | | Formulation D | | |
|---|---|---|---|---|---|---|---|---|---|---|---|
| | | Time | Lower | | Upper | Lower | | Upper | Lower | | Upper |
| Condition | Degradants | (month) | 95% Cl | Median | 95% Cl | 95% Cl | Median | 95% Cl | 95% Cl | Median | 95% Cl |
| 5° C. | RRT 0.52 | 12 | 0.03 | 0.04 | 0.06 | 0.01 | 0.02 | 0.03 | 0.02 | 0.03 | 0.06 |
| (2-8° C.) | | 24 | 0.03 | 0.04 | 0.08 | 0.02 | 0.03 | 0.05 | 0.03 | 0.05 | 0.11 |
| | | 36 | 0.04 | 0.05 | 0.11 | 0.02 | 0.04 | 0.08 | 0.03 | 0.06 | 0.15 |
| | RRT 0.61 | 12 | 0.00 | 0.01 | 0.04 | 0.00 | 0.00 | 0.03 | 0.00 | 0.00 | 0.03 |
| | | 24 | 0.01 | 0.02 | 0.07 | 0.00 | 0.01 | 0.06 | 0.00 | 0.01 | 0.06 |
| | | 36 | 0.01 | 0.03 | 0.11 | 0.00 | 0.01 | 0.08 | 0.00 | 0.01 | 0.09 |
| | RRT 0.66 | 12 | 0.04 | 0.04 | 0.04 | 0.05 | 0.05 | 0.05 | 0.08 | 0.08 | 0.08 |
| | | 24 | 0.04 | 0.04 | 0.04 | 0.05 | 0.05 | 0.05 | 0.08 | 0.08 | 0.08 |
| | | 36 | 0.04 | 0.04 | 0.05 | 0.05 | 0.05 | 0.06 | 0.09 | 0.08 | 0.08 |
| | RRT 0.74 | 12 | 0.00 | 0.00 | 0.01 | 0.00 | 0.00 | 0.00 | 0.00 | 0.00 | 0.00 |
| | | 24 | 0.00 | 0.00 | 0.02 | 0.00 | 0.00 | 0.00 | 0.00 | 0.00 | 0.00 |
| | | 36 | 0.00 | 0.01 | 0.02 | 0.00 | 0.00 | 0.00 | 0.00 | 0.00 | 0.01 |
| | RRT 0.88 | 12 | 0.05 | 0.06 | 0.16 | 0.07 | 0.10 | 0.25 | 0.07 | 0.08 | 0.17 |
| | (CC-06) | 24 | 0.05 | 0.08 | 0.26 | 0.08 | 0.12 | 0.43 | 0.07 | 0.10 | 0.28 |
| | | 36 | 0.06 | 0.09 | 0.37 | 0.08 | 0.14 | 0.61 | 0.08 | 0.11 | 0.37 |
| | RRT 0.95 | 12 | 0.06 | 0.06 | 0.06 | 0.06 | 0.06 | 0.06 | 0.06 | 0.06 | 0.06 |
| | | 24 | 0.06 | 0.06 | 0.06 | 0.06 | 0.06 | 0.06 | 0.06 | 0.06 | 0.06 |
| | | 36 | 0.06 | 0.06 | 0.06 | 0.06 | 0.06 | 0.06 | 0.06 | 0.06 | 0.06 |

Abbreviations:
Cl, confidence limit;
RH, relative humidity;
RRT, relative retention time

TABLE 23
Prediction of Degradant Profile at 25° C./60% RH Conditions for Storage up to 12 Months

| | | | Formulation B | | | Formulation C | | | Formulation D | | |
|---|---|---|---|---|---|---|---|---|---|---|---|
| | | Time | Lower | | Upper | Lower | | Upper | Lower | | Upper |
| Condition | Degradants | (month) | 95% Cl | Median | 95% Cl | 95% Cl | Median | 95% Cl | 95% Cl | Median | 95% Cl |
| 25° C./60% | RRT 0.52 | 6 | 0.04 | 0.06 | 0.09 | 0.03 | 0.05 | 0.07 | .05 | 0.07 | 0.12 |
| RH | | 12 | 0.06 | 0.09 | 0.15 | 0.05 | 0.08 | 0.12 | 0.07 | 0.12 | 0.22 |
| | RRT 0.61 | 6 | 0.01 | 0.03 | 0.10 | 0.00 | 0.01 | 0.08 | 0.00 | 0.02 | 0.07 |
| | | 12 | 0.02 | 0.05 | 0.19 | 0.00 | 0.03 | 0.16 | 0.01 | 0.04 | 0.14 |
| | RRT 0.66 | 6 | 0.04 | 0.05 | 0.06 | 0.05 | 0.06 | 0.07 | 0.08 | 0.09 | 0.10 |
| | | 12 | 0.04 | 0.06 | 0.11 | 0.06 | 0.08 | 0.11 | 0.09 | 0.10 | 0.14 |
| | RRT 0.74 | 6 | 0.01 | 0.03 | 0.07 | 0.00 | 0.01 | 0.02 | 0.01 | 0.01 | 0.03 |
| | | 12 | 0.04 | 0.08 | 0.17 | 0.01 | 0.03 | 0.06 | 0.02 | 0.04 | 0.08 |
| | RRT 0.88 | 6 | 0.09 | 0.18 | 0.47 | 0.12 | 0.24 | 0.71 | 0.12 | 0.24 | 0.60 |
| | (CC-06) | 12 | 0.15 | 0.34 | 0.92 | 0.18 | 0.45 | 1.41 | 0.19 | 0.45 | 1.28 |
| | RRT 0.95 | 6 | 0.06 | 0.06 | 0.07 | 0.06 | 0.06 | 0.06 | 0.06 | 0.06 | 0.06 |
| | | 12 | 0.06 | 0.06 | 0.08 | 0.06 | 0.06 | 0.07 | 0.06 | 0.06 | 0.07 |

Abbreviations:
Cl, confidence limit;
RH, relative humidity;
RRT, relative retention time

Key degradants RRT 0.52, RRT 0.61, RRT 0.66, RRT 0.74, RRT 0.88 (CC-06), and RRT 0.95 were modeled and used to extrapolate shelf-life estimates. The term “RRT” refers to relative retention time. At 5° C. storage condition, no discernible differences in the degradants' levels were observed among the three formulations except for the RRT 0.88 (CC-06) degradant. For all three lead formulations, the unspecified degradant levels were predicted to be within the ICH specification limit (0.15%) after 36-month storage at 5° C. (Table 9). In contrast to formulation B and formulation D, higher levels of RRT0.88 (CC-06) were predicted after 36 months storage at 5° C. for formulation C.

A similar prediction trend was observed at the 25° C./60% RH condition. The model predicted higher levels of RRT 0.88 (CC-06) formation after 12 months storage at 25° C./60% RH for formulation C) in comparison to formulation A and formulation C. The term “RH” as used herein refers to relative humidity.

Overall, based on the ASAP predictions, formulation B and formulation D showed slightly better stability profiles in comparison to the formulation C

### Stability Assessment at ICH Conditions

Furthermore, for all three lead formulations, a stability study was conducted at ICH conditions including 2-8° C. and 25° C./60% RH for up to 6 months. No considerable change in any of the drug product quality attributes were observed for up to 6 months at 2-8° C. and 25° C./60% RH. At 25° C./60% RH, a slight increase in the level of RRT 0.88 (CC-06) was observed for all three lead prototype formulations; however, its level was found to be well below the specification limit. Hence, based on the stability results, it was inferred that all three lead formulations are found to be stable for up to 6 months at 2-8° C. and 25° C./60% RH.

In conclusion, based on the ASAP modeling, it was predicted that the formulation with sodium metabisulfite did not improve drug product stability and is expected to produce higher levels of RRT 0.88 (CC-06) at 2-8° C. and 25° C./60% RH conditions.

### Example 8. In Vitro Dissolution of the Lead Prototype Formulation

Dissolution studies were conducted on the formulations, Formulation B and Formulation D, using a USP dissolution apparatus II (paddle) at 75 rpm rotation speed and 37° C. temperature and 900 mL of pH 6.8 phosphate buffer. Moreover, the in-vitro dissolution of a reference sepiapterin formulation (Formulation A described in Example 1, above) was performed (175 mg dose in 500 mL) in pH 6.8 phosphate buffer using a USP dissolution apparatus II (Paddle) at 50 rpm of rotation speed maintained at a temperature of 37° C.

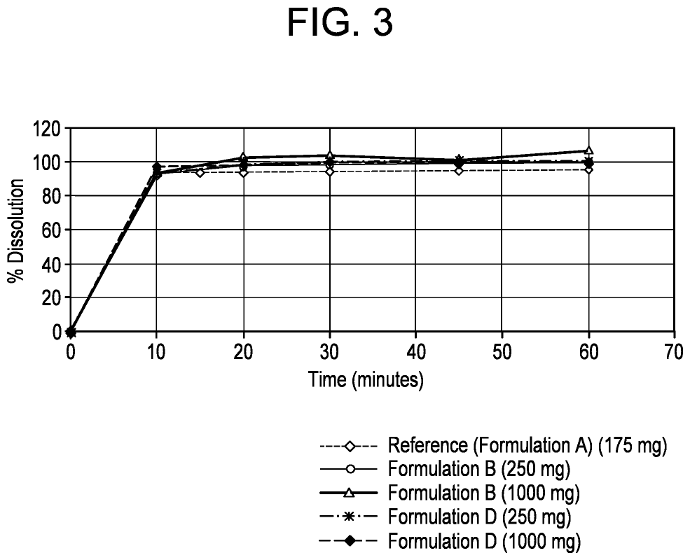

In-vitro dissolution profiles of the reference formulation and formulations of the invention in phosphate buffer pH 6.8 is presented in FIG. 3.

Based on the dissolution profile presented in FIG. 3, it is evident that both the formulations of the invention and the reference formulation exhibited similar rapid dissolution profiles with more than 85% drug dissolved within 15 minutes.

### Example 9. Determination of the Pharmacokinetics of Different Sepiapterin Oral Formulations After a Single Oral Dose to Non-Naïve Male Monkeys

The relative bioavailabilities of two sepiapterin formulations of the invention (formulations B and D) to a reference sepiapterin formulation (reference) were assessed in adult non-naïve male cynomolgus monkeys following a single oral dose at 50 mg/kg Four animals received sequentially the reference formulation (Formulation A) prepared as suspension in Medisca Oral Mix and formulations B and D prepared as suspension in deionized water with a minimum 5-day washout in between. Serial blood samples were collected up to 24 hours postdose. The baseline plasma sepiapterin concentrations (predose) were all BLQ (LLOQ 11.1 ng/ml). After oral administration of the pharmaceutical composition of sepiapterin, sepiapterin was quickly absorbed and converted to BH₄. The plasma sepiapterin concentration was BLQ (LLOQ 11.1 ng/ml) at all time points sampled. The BH₄ plasma concentration at predose was either BLQ (LLOQ 11.1 ng/ml) or just slightly above it (when measurable, it was between 12.8 to 26.5 ng/ml). Plasma BH₄ concentrations reached the maximum approximately 2 hours postdose (Tₘₐₓ) for all 3 formulations. T_{1/2} between 3.21 to 4.2 hours was observed for the 3 formulations. The group mean of uncorrected (of baseline) Cₘₐₓ and AUC₀₋ₗₐₛₜ of BH₄ for formulation B, formulation D, and reference formulation were 908, 893, and 715 ng/ml and 4540, 4270, and 4240 μg·h/mL, respectively. The mean ratios of formulations B and D to the reference for Cₘₐₓ were 127% and 125%, respectively. The mean ratios of formulations B and D to the reference for AUC₀₋ₗₐₛₜ were 106% and 100%, respectively.

### Example 10. Determination of the Pharmacokinetics of Different Sepiapterin Oral Formulations After a Single Oral Dose to Non-Naïve Male Dogs

The relative bioavailabilities of 2 sepiapterin formulations of the invention (Formulations B and D) to the reference formulation were assessed in adult non-naïve male beagle dogs following a single oral dose at 30 mg/kg. Four animals received sequentially the reference formulation (Formulation A) prepared as suspension in Medisca Oral Mix and B and D prepared as suspension in deionized water with a minimum 4-day washout in between. Serial blood samples were collected up to 24 hours postdose. The baseline plasma sepiapterin concentrations (predose) were all below the limit of quantitation (BLQ; LLOQ 11.1 ng/ml). After oral administration, sepiapterin was quickly absorbed and plasma concentrations reached the maximum (Tₘₐₓ) approximately 1-hour postdose for formulations A and C and 2 hours postdose for the reference formulation. T_{1/2} was observed for 3 formulations with the means ranged from 1.6 to 3.4 hours. By 24 hours postdose, plasma sepiapterin concentration were all below the limit of quantitation (BLQ), except one animal with concentration slightly above the LLOQ (15.9 ng/ml). The mean Cₘₐₓ and AUC₀₋ₗₐₛₜ of sepiapterin for formulation B, formulation D, and the reference formulation were 967, 746, and 808 ng/ml and 2210, 2960, and 2470 h×ng/ml, respectively. The mean ratios of formulations B and D to the reference for Cₘₐₓ were 120% and 92.3%, respectively. The mean ratios of formulations B and D to the reference for AUC₀₋ₗₐₛₜ were 89.5% and 120%, respectively.

The BH₄ plasma concentration was measurable at predose in the range from 14.3 to 20.1 ng/ml. After the pharmaceutical composition of sepiapterin administration, the sepiapterin in the pharmaceutical composition of sepiapterin was quickly converted to BH₄ and plasma BH₄ concentrations reached the maximum between 2 to 4 hours postdose (Tₘₐₓ) for all 3 formulations. The T_{1/2} was similar for 3 formulations with the group mean ranging from 6.08 to 7.01 hours. The group mean of uncorrected (of baseline) Cₘₐₓ and AUC_{0-24 h} of BH₄ were 867, 861, and 1010 ng/ml and 5980, 7080, and 6920 μg×h/mL for formulation B, formulation D, and the reference formulation, respectively. The mean ratios of formulations B and C to the reference for Cₘₐₓ were 86% and 85%, respectively. The mean ratios of formulations B and C to the reference for AUC_{0-24 h} were 86% and 102%, respectively.

### Example 11. Development Stability Study

Formulation B was packaged in heat sealed aluminum sachets in 250 mg and 1000 mg strength configurations and its stability assessments were conducted at ICH conditions including 2-8° C. and 25° C./60% RH. The stability data of the formulation, B at 2-8° C. and 25° C./60% RH for up to 18 months for 250 mg and up to 12 months for 1000 mg are provided in Table 24 through Table 27.

TABLE 24
Stability Data for Formulation B, 250 mg at 2-8° C.

| Parameter | Draft | 2-8° C. | | | | | | |
|---|---|---|---|---|---|---|---|---|
| Tested | Specification | Initial | 2 Months | 3 months | 6 Months | 9 Months | 12 M | 18 M |
| Appearance | For | Yellow | Yellow | Yellow | Yellow | Yellow | Yellow | Yellow |
| | information | Granules | Granules | Granules | Granules | Granules | Granules | Granules |
| ADPW | For | 1016.03 | 1018.62 | 1014.87 | 1009.93 | 1011.88 | 1001.33 | 997.84 |
| | information | | | | | | | |
| | (mg) | | | | | | | |
| Water | For | 3.42 | 3.24 | 3.07 | 3.20 | 3.02 | 3.21 | 3.06 |
| content | information | | | | | | | |
| | (%) | | | | | | | |
| Identity | Complies | Complies | NT | NT | NT | NT | NT | NT |
| Assay | 90-110% label | 100.4 | 100.7 | 99.4 | 100.3 | 99.1 | 98.3 | 97.9 |
| | claim [%] | | | | | | | |
| Uniformity | Complies with | AV = 4.0 | NT | NT | NT | NT | NT | NT |
| of Dosage | USP <905> | | | | | | | |
| forms | AV ≤15.0 | | | | | | | |
| Purity | For | RRT | RRT | RRT | RRT | RRT | RRT | RRT |
| | Information | 0.49 <0.05 | 0.51: <0.05 | 0.49: <0.05 | 0.49: <0.05 | 0.51: <0.05 | 0.49: <0.05 | 0.49: <0.05 |
| | [%] | RRT | RRT | RRT | RRT | RRT | RRT | RRT |
| | (report with | 0.61: 0.10 | 0.62: <0.05 | 0.60: <0.05 | 0.61: 0.05 | 0.62: <0.05 | 0.61: <0.05 | 0.61: 0.05 |
| | RRT) | 6-LP: 0.10 | 6-LP: 0.09 | 6-LP: 0.12 | 6-LP: 0.13 | 6-LP: 0.14 | 6-LP: 0.11 | 6-LP: 0.17 |
| | | RRT | RRT0.94: <0.05 | Total: 0.12 | Total: 0.18 | Total: 0.14 | Total: 0.11 | Total: 0.22 |
| | | 0.92: 0.05 | Total: 0.09 | | | | | |
| | | Total: 0.25 | | | | | | |
| Dissolution | Profile for | 10 min: 97% | 10 min: 98% | 10 min: 101% | 10 min: 103% | 10 min: 92% | 10 min: 88% | 10 min: 91% |
| | information | 20 min: 98% | 20 min: 98% | 20 min: 101% | 20 min: 103% | 20 min: 97% | 20 min: 89% | 20 min: 90% |
| | | 30 min: 98% | 30 min: 98% | 30 min: 100% | 30 min: 102% | 30 min: 97% | 30 min: 89% | 30 min: 88% |
| | | 45 min: 98% | 45 min: 99% | 45 min: 100% | 45 min: 102% | 45 min: 97% | 45 min: 89% | 45 min: 89% |
| | | 60 min: 99% | 60 min: 98% | 60 min: 100% | 60 min: 102% | 60 min: 97% | 60 min: 88% | 60 min: 99% |
| Polymorphism | For | Form F | NT | NT | NT | NT | NT | NT |
| | information | | | | | | | |

Abbreviations:
NT; not tested,
RRT; relative retention time

TABLE 25
Stability Data for Formulation B, 250 mg at 25° C./60% RH

| Parameter | Draft | 25° C./60% RH | | | | | | |
|---|---|---|---|---|---|---|---|---|
| Tested | Specification | Initial | 2 Months | 3 months | 6 Months | 9 Months | 12 M | 18 M |
| Appearance | For | Yellow | Yellow | Yellow | Yellow | Yellow | Yellow | Yellow |
| | information | Granules | Granules | Granules | Granules | Granules | Granules | Granules |
| ADPW | For | 1016.03 | 1009.58 | 1014.98 | 1012.77 | 1033.50 | 1011.03 | 1010.84 |
| | information | | | | | | | |
| | (mg) | | | | | | | |
| Water content | For | 3.42 | 3.35 | 3.13 | 3.10 | 3.13 | 3.43 | 3.23 |
| | information | | | | | | | |
| | (%) | | | | | | | |
| Identity | Complies | Complies | NT | NT | NT | NT | NT | NT |
| Assay | 90-110% | 100.4 | 98.7 | 99.0 | 98.9 | 98.5 | 97.9 | 98.1 |
| | label claim | | | | | | | |
| | [%] | | | | | | | |
| Uniformity of | Complies | AV = 4.0 | NT | NT | NT | NT | NT | NT |
| Dosage forms | with | | | | | | | |
| | USP <905> | | | | | | | |
| | AV ≤15.0 | | | | | | | |
| Purity | For | RRT | RRT0.51: <0.05 | RRT | RRT | RRT | RRT | RRT |
| | Information | 0.49 <0.05 | RRT | 0.49: <0.05 | 0.49: 0.07 | 0.51: 0.07 | 0.50: 0.11 | 0.48: 0.09 |
| | [%] | RRT | 0.62: 0.05 | RRT | RRT | RRT | RRT | RRT |
| | (report | 0.61: 0.10 | 6-LP: 0.15 | 0.60: 0.06 | 0.61: 0.11 | 0.62: 0.07 | 0.61: <0.05 | 0.61: 0.07 |
| | with RRT) | 6-LP: 0.10 | RRT0.92: <0.05 | 6-LP: 0.19 | 6-LP: 0.32 | 6-LP: 0.36 | 6-LP: 0.33 | 6-LP: 0.43 |
| | | RRT | Total: 0.20 | Total: 0.25 | Total: 0.50 | Total: 0.50 | Total: 0.44 | Total: 0.59 |
| | | 0.92: 0.05 | | | | | | |
| | | Total: 0.25 | | | | | | |
| Dissolution | Profile for | 10 min: 97% | 10 min: 98% | 10 min: 98% | 10 min: 99% | 10 min: 94% | 10 min: 88% | 10 min: 92% |
| | information | 20 min: 98% | 20 min: 99% | 20 min: 98% | 20 min: 100% | 20 min: 96% | 20 min: 88% | 20 min: 90% |
| | | 30 min: 98% | 30 min: 99% | 30 min: 98% | 30 min: 100% | 30 min: 96% | 30 min: 88% | 30 min: 88% |
| | | 45 min: 98% | 45 min: 100% | 45 min: 98% | 45 min: 100% | 45 min: 96% | 45 min: 88% | 45 min: 88% |
| | | 60 min: 99% | 60 min: 100% | 60 min: 98% | 60 min: 100% | 60 min: 96% | 60 min: 88% | 60 min: 87% |
| Polymorphism | For | Form F | NT | NT | NT | NT | NT | NT |
| | information | | | | | | | |

Abbreviations:
NT; not tested,
RRT; relative retention time

TABLE 26
Stability Data for Formulation B, 1000 mg at 2-8° C.

| Parameter | | 2-8° C. | | | | | |
|---|---|---|---|---|---|---|---|
| Tested | Specification | Initial | 1 Months | 3 months | 6 Months | 9 Months | 12 Months |
| Appearance | Yellow to | Corresponds | Corresponds | Corresponds | Corresponds | Corresponds | Corresponds |
| | orange | | | | | | |
| | powder | | | | | | |
| ADPW | For | 4086.4 | 4038.8 | 4060.0 | 4023.7 | 4057.6 | 4092.5 |
| | information | | | | | | |
| | (mg) | | | | | | |
| Water | ≤7.0 (%) | 3.5 | 3.7 | 3.4 | 3.5 | 3.6 | 3.5 |
| content | | | | | | | |
| Assay | 90-110% | 101.2 | 100.8 | 101.4 | 102.3 | 97.8 | 100.7 |
| | label claim | | | | | | |
| | [%] | | | | | | |
| Impurities | Individual | <0.05 | <0.05 | <0.05 | <0.05 | <0.05 | RRT |
| and | Unspecified: ≤0.15% | 0.08 | 0.12 | 0.07 | 0.07 | 0.11 | 0.52: 0.05 |
| Degradation | CC-06: ≤0.65% | 0.08 | 0.12 | 0.07 | 0.07 | 0.11 | 0.26 |
| Products | Total ≤3.0% | | | | | | 0.31 |
| Dissolution | NLT 80% | 99 | 105 | 97 | 100 | 95 | 100 |
| PTC923 | (Q = 75%) | (91, 97, | (86, 99, | (88, 94, | (88, 95, | (89, 93, | (96, 101, |
| (Sepiapterin) | after30 min | 99, 100, | 105, 104, | 97, 99, | 100, 100, | 95, 97, | 100, 108, |
| | (Record % | 101) | 102) | 99) | 100) | 98) | 109) |
| | dissolved at | | | | | | |
| | 10, 20, 30, | | | | | | |
| | 45, 60 min) | | | | | | |
| Microbiology | ≤10³ CFU/g | <5 | NT | NT | NT | NT | NT |
| TAMC | ≤10² CFU/g | <5 | | | | | |
| TYMC | Absence/g | absence | | | | | |
| E. coli | | | | | | | |

Abbreviations:
NT; not tested,
RRT; relative retention time

TABLE 27
Stability Data for Formulation B, 1000 mg at 25°/60% RH

| Parameter | | 25° C./60% RH | | | | | | |
|---|---|---|---|---|---|---|---|---|
| Tested | Specification | Initial | 1 Months | 2 months | 3 Months | 6 Months | 9 Months | 12 Months |
| Appearance | Yellow to | Corresponds | Corresponds | Corresponds | Corresponds | Corresponds | Corresponds | Corresponds |
| | orange | | | | | | | |
| | powder | | | | | | | |
| ADPW | For | 4086.4 | 4060.0 | 4093.0 | 4055.0 | 4082.0 | 4067.2 | 4062.3 |
| | information | | | | | | | |
| | (mg) | | | | | | | |
| Water | ≤7.0 (%) | 3.5 | 4.0 | 3.4 | 3.5 | 3.2 | 3.6 | 3.4 |
| content | | | | | | | | |
| Assay | 90-110% | 101.2 | 100.8 | 102.3 | 99.6 | 101.8 | 98.2 | 98.8 |
| | label claim | | | | | | | |
| | [%] | | | | | | | |
| Impurities | Individual | <0.05 | RRT | RRT | RRT | RRT | RRT | RRT |
| and | Unspecified: ≤0.15% | 0.08 | 0.48: 0.06 | 0.48: 0.06 | 0.49: 0.05 | 0.50: 0.06 | 0.49: 0.09 | 0.52: 0.11 |
| Degradation | CC-06: ≤0.65% | 0.08 | 0.21 | RRT | 0.19 | RRT | RRT | 0.47 |
| Products | Total ≤3.0% | | 0.27 | 0.60: 0.08 | 0.24 | 0.61: 0.06 | 0.61: 0.05 | 0.58 |
| | | | | 0.26 | | 0.30 | 0.36 | |
| | | | | 0.40 | | 0.42 | 0.50 | |
| Dissolution | NLT 80% | 99 | 98 | 97 | 98 | 97 | 97 | 102 |
| PTC923 | (Q = 75%) | (91, 97, | (94, 98, | (85, 91, | (86, 97, | (86, 93, | (95, 96, | (96, 100, |
| (Sepiapterin) | after30 min | 99, 100, | 98, 100, | 97, 99, | 98, 100, | 97, 99, | 97, 98, | 102, 101, |
| | (Record % | 101) | 101) | 101) | 100) | 99) | 97) | 102) |
| | dissolved at | | | | | | | |
| | 10, 20, 30, | | | | | | | |
| | 45, 60 min) | | | | | | | |
| Microbiology | ≤10³ CFU/g | <5 | NT | NT | NT | NT | NT | NT |
| TAMC | ≤10² CFU/g | <5 | | | | | | |
| TYMC | Absence/g | absence | | | | | | |
| E. coli | | | | | | | | |

Abbreviations:
NT; not tested,
RRT; relative retention time

### Example 12—Evaluation of Relative Oral Bioavailability of Two Formulations

A Phase 1, randomized, open-label crossover study was conducted in 18 human subjects to evaluate the relative oral bioavailability of Formula A (comparator from Phase I study described in Example 1) and Formula B (see Example 7, above), as follows. The primary objective was to assess the relative bioavailability of BH₄ and sepiapterin following administration of two dose levels (20 and 60 mg/kg/day) of each of the two formulations under fed conditions, a low-fat diet in healthy adult human subjects. Secondary objectives were to assess the safety and tolerability of the two formulations at the two different dose levels and to assess the palatability of Formulation B.

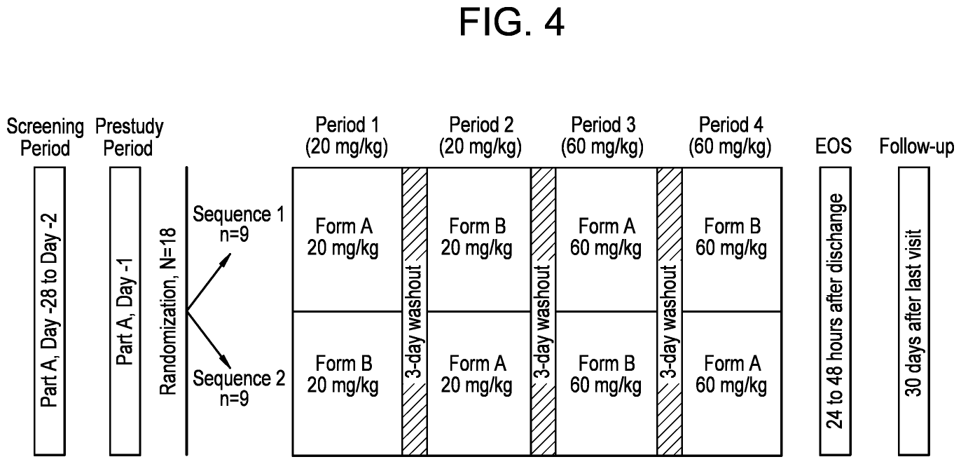

Subjects participating in the study were randomized into one of two treatment sequences (Sequence 1 or Sequence 2) including 20 and 60 mg/kg of each of Formulations A and B in a low-fat fed state during the in-clinic period (Day 1 through Day 11). Subjects received a single oral dose of the assigned study treatment on Days 1, 4, 7 and 10 while on a low-fat diet. There was a washout of three days between doses. For each study drug administration, blood samples were collected predose and for up to 24 hours postdose for pharmacokinetic (PK) assessment of analytes BH₄ and sepiapterin. A Study Schema of the study is illustrated in FIG. 4.

Palatability assessments were performed after administration of each of the study treatments. Safety was evaluated by symptom-driven physical examinations, vital signs assessment, 12-lead ECGs, routine clinical assessments, and adverse event assessments.

Subjects in the study all met the following inclusion criteria: They were males or females aged between 18 and 55 years old and had a Body Mass Index between 18.5 and 30.0 kg/m². Women of childbearing potential had to have a negative pregnancy testing screening.

Formula A was suspended in Medisca Oral Mix prior to administration, while Formula B was suspended in water prior to administration. Each subject received a single dose of the following:
Formulation A suspension, sepiapterin dose of 20 mg/kg
Formulation A suspension, sepiapterin dose of 60 mg/kg
Formulation B suspension, sepiapterin dose of 20 mg/kg
Formulation B suspension, sepiapterin dose of 60 mg/kg

All doses were administered in a low-fat fed condition, following an overnight fast of 10 hours and within 30 minutes after a standard low-fat breakfast (FDA-defined) with up to 240 ml of water. Subjects were permitted to consume a snack 4 hours postdose if desired.

### Criteria Evaluated:

The following pharmacokinetics parameters were to be calculated for BH₄ and sepiapterin in plasma: area under the concentration versus time curve (AUC) from time zero to the last non-zero concentration, (AUC0-t), AUC from time zero to infinity (AUC0-inf), percent of AUC0-inf extrapolated (AUC % extrap), Cmax, Tmax, T1/2, elimination rate constant (Kel), apparent total plasma clearance (CL/F), and apparent volume of distribution (Vz/F).

The PK parameters for BH₄ were derived from both baseline corrected and uncorrected data.

A mixed effect analysis of variance model is to be performed on the natural log-transformed AUC0-t, AUC0-inf, and Cₘₐₓ of BH₄ and sepiapterin with formulation and sequence as fixed effects and subject as a random effect. Tₘₐₓ will not be log-transformed.

### Results

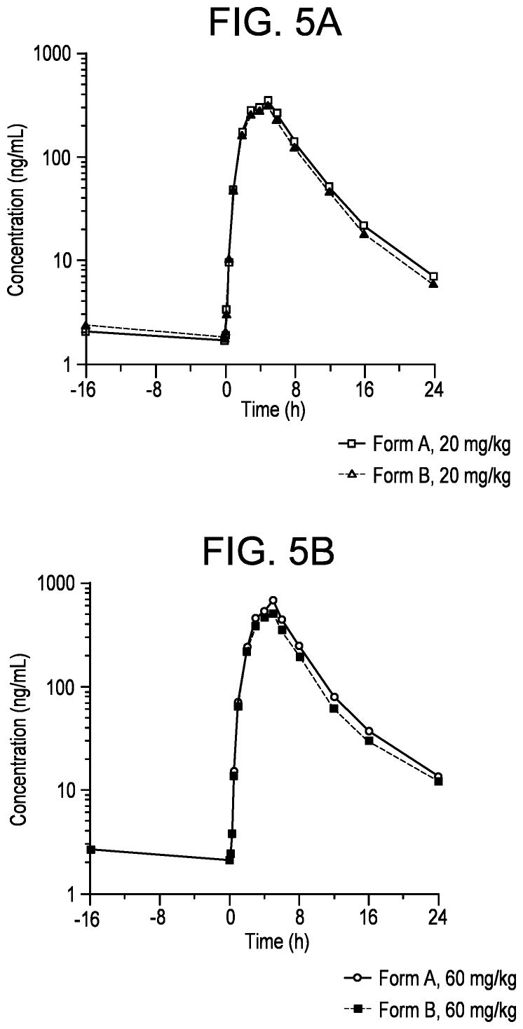

Preliminary results from measurement of BH₄ levels in the study described immediately above, without baseline correction are illustrated in FIGS. 5A and 5B and summarized in Table 28, below. Specifically, geometric least square mean data obtained from measurements of BH₄ from administration of 20 mg/kg doses of sepiapterin from Formula A (open squares) and B (solid triangles) are illustrated in FIG. 5A. Geometric least square mean measurements of BH₄ form administration of 60 mg/kg doses of sepiapterin from Formula A (open circles) and B (solid squares) are illustrated in FIG. 5B.

TABLE 28
BH₄ Data Summary Without Baseline Correction

| Dose | | Form A | Form B | |
|---|---|---|---|---|
| (mg/kg) | Parameter | (Ref) | (Test) | Ratio % (90% CI) |
| 20 | AUCinf | 2151 | 1936 | 89.977 (83.779-96.633) |
| 20 | AUClast | 2123 | 1910 | 89.984 (83.799-96.625) |
| 20 | Cmax | 341 | 304 | 89.174 (79.88-99.549) |
| 60 | AUCinf | 3530 | 3067 | 86.883 (79.683-94.733) |
| 60 | AUClast | 3471 | 3013 | 86.806 (79.603-94.662) |
| 60 | Cmax | 580 | 502 | 86.655 (77.529-96.855) |

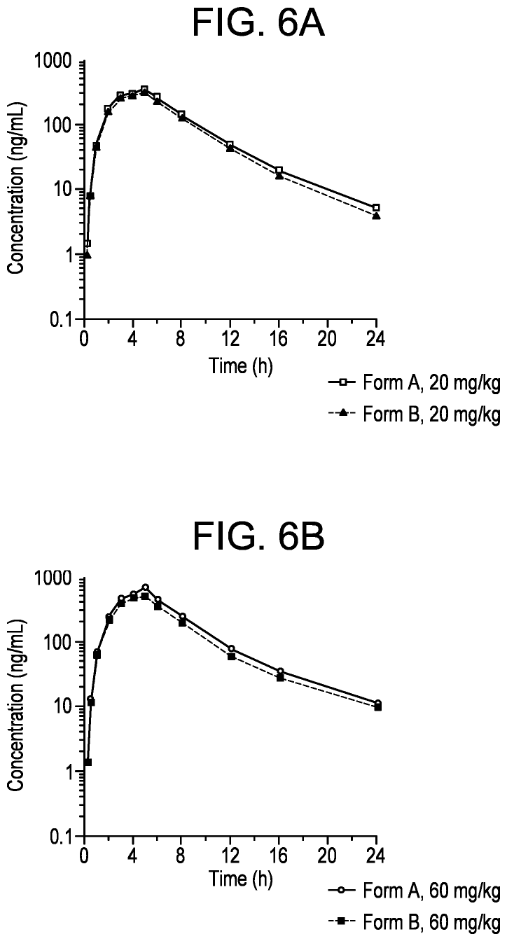

Preliminary results from measurement of BH₄ levels in the study described immediately above, with baseline correction are illustrated in FIGS. 6A and 6B and summarized in Table 29, below. Specifically, the geometric least square mean data obtained from measurements of BH₄ from administration of 20 mg/kg doses of sepiapterin from Formula A (open squares) and B (solid triangles) are illustrated in FIG. 5A. Geometric least square mean measurements of BH₄ form administration of 60 mg/kg doses of sepiapterin from Formula A (open circles) and B (solid squares) are illustrated in FIG. 5B.

TABLE 29
BH₄ Data Summary with Baseline Correction:

| Dose | | Form A | Form B | |
|---|---|---|---|---|
| (mg/kg) | Parameter | (Ref) | (Test) | Ratio % (90% CI) |
| 20 | AUCinf | 2095 | 1875 | 89.504 (83.243-96.236) |
| 20 | AUClast | 2075 | 1859 | 89.588 (83.336-96.309) |
| 20 | Cmax | 340 | 302 | 89.071 (79.779-99.445) |
| 60 | AUCinf | 3456 | 2992 | 86.569 (79.359-94.435) |
| 60 | AUClast | 3411 | 2952 | 86.527 (79.3-94.413) |
| 60 | Cmax | 577 | 500 | 86.599 (77.453-96.825) |

In both Tables 28 and 29 and in FIGS. 5 and 6, one can see that the mean ratios for each factor measured were lower with Formula B than with Formula A. However one can also see that the mean results were all at least 85% of what was obtained from Formula A, indicating the two formula have very similar PK properties. Other embodiments are in the claims.

What is claimed is:

1. A solid pharmaceutical composition comprising sepiapterin, a water-soluble diluent, a water-insoluble diluent, a disintegrant, a suspending agent, a glidant, a sweetener, and a lubricant.

2. The pharmaceutical composition of claim 1, wherein the sepiapterin is crystalline.

3. The pharmaceutical composition of claim 2, wherein the crystalline sepiapterin is crystalline Form F.

4. The pharmaceutical composition of claim 2, wherein the crystalline sepiapterin is crystalline Form D.

5. The pharmaceutical composition of claim 2, wherein the crystalline sepiapterin is crystalline Form A, B, C, E, or G.

6. The pharmaceutical composition of claim 1, wherein the pharmaceutical composition is a powder.

7. The pharmaceutical composition of any one of claims 1 to 6, wherein the pharmaceutical composition comprises less than 60% humidity.

8. The pharmaceutical composition of any one of claims 1 to 7, wherein the pharmaceutical composition is packaged in a sachet.

9. The pharmaceutical composition of any one of claims 2 to 8, wherein the pharmaceutical composition comprises about 20%-50% sepiapterin by weight.

10. The pharmaceutical composition of claim 9, wherein the pharmaceutical composition comprises about 25% sepiapterin by weight.

11. The pharmaceutical composition of any one of claims 1 to 10, wherein the pharmaceutical composition comprises multiple diluents selected from lactose, glucose, mannitol, xylitol, maltitol, sorbitol, isomalt, crystalline cellulose, sucrose, fructose, maltose, trehalose, and microcrystalline cellulose.

12. The pharmaceutical composition of any one of claims 1 to 11, wherein at least one diluent is soluble in water and at least one diluent is insoluble in water.

13. The pharmaceutical composition of claim 12, wherein the pharmaceutical composition comprises about 5 to 65% water-soluble diluent by weight and about 15 to 40% water-insoluble diluent by weight.

14. The pharmaceutical composition of claim 13, wherein is the water-soluble diluents are isomalt and mannitol.

15. The pharmaceutical composition of claim 14, wherein the composition comprises about 40% isomalt by weight and about 10% mannitol by weight.

16. The pharmaceutical composition of claim 12, wherein one water-insoluble diluent water-insoluble diluent is microcrystalline cellulose.

17. The pharmaceutical composition of claim 15, wherein the composition comprises about 21% microcrystalline cellulose by weight.

18. The pharmaceutical composition of any one of claims 1 to 17, wherein the disintegrant is selected from sodium starch glycolate, crospovidone, cross-linked alginic acid, crosslinked starch, crosslinked alginate sodium, carmellose, carmellose calcium, croscarmellose sodium, glycerin fatty acid ester, low-substituted sodium carboxymethyl starch and partially pregelatinized starch.

19. The pharmaceutical composition of claim 18, wherein the composition comprises about 0.5 to 1.5% disintegrant by weight.

20. The pharmaceutical composition of claim 18, wherein the disintegrant is croscarmellose sodium, and the composition comprises about 1.0% croscarmellose sodium by weight.

21. The pharmaceutical composition of any one of claims 1 to 20, wherein the glidant is selected from include colloidal silicon dioxide, hydrated sodium sulfoaluminate and talc.

22. The pharmaceutical composition of claim 21, wherein the composition comprises about 0.2 to 0.6% glidant by weight.

23. The pharmaceutical composition of claim 22, wherein the glidant is colloidal silicon dioxide, and the composition comprises about 0.4% colloidal silicon dioxide by weight.

24. The pharmaceutical composition of any one of claims 1 to 23, wherein the sweetener is selected from sucralose, sodium saccharin, aspartame, and neutrame.

25. The pharmaceutical composition of claim 24, wherein the sweetener is sucralose, wherein the pharmaceutical composition comprises about 0.5% to about 2.0% sweetener by weight.

26. The pharmaceutical composition of claim 25, wherein the sweetener is sucralose, and the pharmaceutical composition comprises about 1% sucralose by weight.

27. The pharmaceutical composition of any one of claims 1 to 26, wherein the lubricant is selected from glyceryl behenate, glyceryl behaptate; sodium stearyl fumarate, stearic acid, magnesium stearate, calcium stearate, sodium stearate, hydrogenated vegetable oils; colloidal silica; talc; waxes; boric acid; sodium benzoate; sodium acetate; sodium fumarate; sodium chloride; DL-leucine; polyethylene glycol; sodium oleate; sodium lauryl sulfate; and magnesium lauryl sulfate.

28. The pharmaceutical composition of claim 27, wherein the composition comprises about 0.4 to 0.8% lubricant by weight.

29. The pharmaceutical composition of claim 28, wherein the lubricant is magnesium stearate, and the pharmaceutical composition comprises about 0.6% magnesium stearate by weight.

30. The pharmaceutical composition of any one of claims 1 to 29, wherein the suspending agent is selected from xanthan gum and hydroxyethyl cellulose.

31. The pharmaceutical composition of claim 30, wherein the pharmaceutical composition comprises about 0.5 to 2.0% suspending agent by weight.

32. The pharmaceutical composition of claim 31, wherein the suspending agent is xanthan gum, and the pharmaceutical composition comprises about 1% xanthan gum by weight.

33. The pharmaceutical composition of claim 1, wherein the composition comprises about 25% sepiapterin, about 21% microcrystalline cellulose, about 40% isomalt, about 10% mannitol, about 1% croscarmellose sodium, about 1% xanthan gum, about 0.4% colloidal silicon dioxide, about 1.0% sucralose, and about 0.6% magnesium stearate by weight.

34. The pharmaceutical composition of claim 1, wherein the composition further comprises an antioxidant.

35. A method of preparing the pharmaceutical composition of any one of claims 1 to 34 comprising (1) sieving sepiapterin and at least one water-soluble diluent, water-insoluble diluent, disintegrant, suspending agent, glidant, and sweetener, and blending, forming a pre-blended mixture, (2) sieving a lubricant, (3) mixing the lubricant with the preblended mixture to form a lubricated preblended mixture, (4) preparing ribbons of the lubricated preblended mixture using a roller compactor, (5) milling the ribbons to form a milled formulation, (6) blending the milled formulation with a blender, forming the pharmaceutical composition of claim 1.

36. The method of claim 35, further comprising filling the pharmaceutical composition into sachets using a sachet filler.

37. A method of preparing a liquid formulation comprising dispersing the pharmaceutical composition of claims 1 to 34 in a liquid, optionally wherein the liquid is water or fruit juice.

38. The method of claim 37, wherein the liquid formulation is a suspension.
